(19) United States
(12) Patent Application Publication
(10) Pub. No.: US 2016/0159787 A1
(43) Pub. Date: Jun. 9, 2016

(54) Cis-tetrahydro-spiro(cyclohexane-1, 1' -pyrido[3,4-b]indole)-4-amine Compounds

(71) Applicant: Gruenenthal GmbH, Aachen (DE)

(72) Inventors: Klaus LINZ, Wachtberg (DE); Saskia ZEMOLKA, Aachen (DE); Bert NOLTE, Bad Muenstereifel (DE); Stefan SCHUNK, Aachen (DE); Hans SCHICK, Berlin-Weissensee (DE)

(21) Appl. No.: 14/846,155

(22) Filed: Sep. 4, 2015

Related U.S. Application Data
(63) Continuation of application No. 13/192,641, filed on Jul. 28, 2011.
(60) Provisional application No. 61/368,314, filed on Jul. 28, 2010.

(30) Foreign Application Priority Data
Jul. 28, 2010 (EP) 10007822.9

(51) Int. Cl. C07D 471/04

(52) U.S. Cl. CPC C07D 471/04

(57) ABSTRACT

Cis-tetrahydro-spiro(cyclohexane-1,1′-pyrido[3,4-b]indole)-4-amine compounds which act on the nociceptin/ORL-1 receptor system as well as on the μ-opioid receptor system and which are distinguished in particular by selective effectiveness in the treatment of chronic pain, such as inflammatory pain, visceral pain, tumour pain, and neuropathic pain, without at the same time developing pronounced effectiveness against acute, nociceptive pain.

### CROSS REFERENCE TO RELATED APPLICATIONS

This application is a continuation of U.S. application Ser. No. 13/192,641, filed Jul. 28, 2011, which claims priority from U.S. provisional patent application No. 61/368,314, filed Jul. 28, 2010 and European patent application no. EP 10007822.9, also filed Jul. 28, 2010, the entire disclosures of each of which are incorporated herein by reference.

### BACKGROUND OF THE INVENTION

The invention relates to compounds which act on the nociceptin/ORL-1 receptor system as well as on the μ-opioid receptor system and which are distinguished in particular by selective effectiveness in the treatment of chronic pain (inter alia inflammatory pain, visceral pain, tumour pain, preferably neuropathic pain) without at the same time developing pronounced effectiveness in the case of acute, nociceptive pain. The compounds according to the invention are cis-tetrahydro-spiro(cyclohexane-1,1′-pyrido[3,4-b]indole)-4-amine derivatives.

Chronic pain can be divided into two large groups. Pathophysiological nociceptor pain is triggered following tissue traumas by the excitation of intact nociceptors. It includes in particular chronic inflammatory pain. Pain caused by mechanical, metabolic or inflammatory damage to nerves themselves, on the other hand, is referred to as neuropathic pain. The treatment of chronic pain is a major medical challenge because, although some of the medicaments on the market are highly effective in the case of acute pain, they result in many cases in an unsatisfactory treatment of pain in the case of chronic and, in particular, neuropathic pain.

Inflammatory processes belong to the most important mechanisms of pain formation. Typical inflammatory pain is triggered by the release of bradykinin, histamine and prostaglandins with acidification of the tissue and the pressure of the exsudate on the nociceptors. Sensitisation phenomena in the central nervous system frequently occur as a result, which manifest themselves in an increase in spontaneous neuronal activity and in stronger responses of central neurons (Coderre et al., Pain 1993, 52, 259-285). These changes in the response behaviour of central neurons can contribute towards spontaneous pain and hyperalgesia (increased pain sensitivity to a noxious stimulus), which are typical of inflamed tissue (Yaksh et al., PNAS 1999, 96, 7680-7686).

Non-steroidal antiphlogistics (NSAIDs), which also have an antiinflammatory component in addition to the analgesic action, have proved to be particularly successful in the treatment of inflammatory pain (Dickensen, A., International Congress and Symposium Series—Royal Society of Medicine (2000), 246, 47-54). Their use in the long-term therapy of chronic pain is limited, however, by sometimes considerable undesirable effects, such as gastroenteral ulcers or toxic kidney damage. In the case of severe to very severe inflammatory pain (for example within the context of chronic pancreatitis), NSAIDs possibly reduce the pain only slightly but, on account of the increased risk of bleeding, lead to a risk that is too high. The next step is generally treatment with μ-opioids, dependency on narcotics being widespread among the persons concerned (Vercauteren et al., Acta Anaesthesiologica Belgica 1994, 45, 99-105). There is therefore an urgent need for compounds which are highly effective in the case of inflammatory pain and possess a reduced dependency potential.

Neuropathic pain occurs when peripheral nerves are damaged in a mechanical, metabolic or inflammatory manner. The pain profiles that occur thereby are characterised predominantly by the appearance of spontaneous pain, hyperalgesia and allodynia (pain is already triggered by non-toxic stimuli) (see Baron, Clin. J. Pain 2000; 16 (2 Suppl), 12-20). The causes and characteristics, and therefore also the treatment needs, of neuropathic pain are many and varied. It occurs as a result of damage to or disease of the brain, spinal cord or peripheral nerves. Possible causes are operations (e.g. phantom pain following amputation), spinal cord injuries, stroke, multiple sclerosis, alcohol or medicament abuse or further toxic substances, cancer, and also metabolic diseases such as diabetes, gout, renal insufficiency or cirrhosis of the liver, as well as infectious diseases (inter alia Herpes zoster, Pfeiffer's glandular fever, ehrlichiosis, typhus, diphtheria, HIV, lues or borreliosis). The pain experience has very different signs and symptoms (e.g. tingling, burning, shooting, electrifying or radiating pain), which can change over time in terms of number and intensity.

The basic pharmacological therapy of neuropathic pain includes tricyclic antidepressants and anticonvulsives, which are used as monotherapy or also in combination with opioids. In most cases, such medicaments bring only a certain degree of pain relief, while freedom from pain is often not achieved. The side-effects that frequently occur often prevent the doses of the medicaments from being increased in order to achieve adequate alleviation of pain. In fact, the satisfactory treatment of neuropathic pain frequently requires a higher dose of a μ-opioid than does the treatment of acute pain, as a result of which the side-effects become even more important. Today, neuropathic pain is therefore difficult to treat. It is only partially alleviated even by high doses of stage-3 opioids (Saudi Pharm. J. 2002, 10 (3), 73-85).

Opioids which are used in the treatment of neuropathic pain are usually also effective against acute pain at the same time. It has hitherto not been possible to separate the treatment of neuropathic pain on the one hand and acute pain on the other hand. Depending on the dose of the opioids, therefore, any pain sensation of the patient is suppressed, which can be wholly disadvantageous. Acute pain has a protective function for the body, which is lost if the sensation of acute pain is impaired or suppressed. There is therefore a need to maintain the general sensation of pain while at the same time controlling neuropathic pain.

Spirocyclic cyclohexane derivatives that act on the nociceptin/ORL-1 and on the μ-opioid receptor system are known in the prior art. These compounds are distinguished inter alia by extraordinarily great structural variability and are suitable inter alia for the treatment of inflammatory and neuropathic pain. In this connection, reference may be made, for example, to the whole of U.S. Pat. No. 7,547,707 (=WO 2004/043967), U.S. Pat. No. 7,288,560 (=WO 2005/063769), U.S. Pat. No. 7,332,519 (=WO 2005/066183) and US 2008/0221141 (=WO 2006/108565).

There is a need for medicaments which are effective in the treatment of chronic, in particular neuropathic, pain and which at the same time affect the perception of acute pain to the smallest possible degree. Where possible, such medicaments should contain such a small dose of active ingredient that satisfactory pain therapy can be ensured without the occurrence of intolerable side-effects.

### SUMMARY OF THE INVENTION

The object underlying the invention is to provide novel compounds which are suitable as medicaments and have advantages over the prior art.

This and other objects have been achieved by the invention as described and claimed hereinafter.

The invention relates to compounds of the general formula (I)

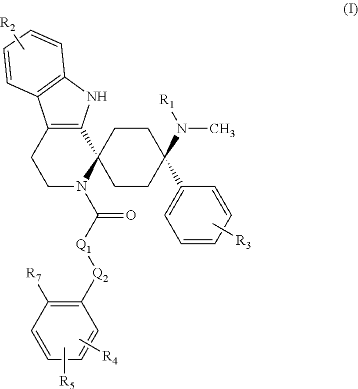

wherein
R₁ is —H or CH₃;
R₂ is —H or -halogen;
R₃ is —H or -halogen;
R₄ is —H, -halogen or —OC₁₋₃-alkyl;
R₅ is —H, -halogen or —OC₁₋₃-alkyl;
-Q₁-Q₂- forms the group —CH₂— or —CR₆═CH—; and
R₆ and R₇ are either both simultaneously —H or together via the bridge —S— form a five-membered ring;
   in the form of the free bases or physiologically acceptable salts.

It has been found, surprisingly, that the compounds according to the invention act on the nociceptin/ORL-1 and on the μ-opioid receptor system and are particularly effective in the treatment of chronic pain, in particular neuropathic pain, without at the same time suppressing the perception of acute pain. Moreover, these compounds surprisingly exhibit—if at all—only very slight opioid-typical side-effects in the analgesically effective dose range.

The compounds according to the invention exhibit very high analgesic effectiveness in the treatment of chronic pain, in particular neuropathic pain, preferably following poly- or mono-neuropathic diseases.

It has been found, surprisingly, that the compounds have no effect on normal nociception in healthy animals or in the healthy tissue of mononeuropathic animals at doses which lead to almost complete elimination of neuropathic pain in mono- or poly-neuropathy models. This means that the compounds eliminate the pathological condition (allodynia or hyperalgesia) but at the same time impair normal pain sensation at most only slightly—if at all. The antinociceptive action of the compounds in acute pain is therefore negligible.

The compounds according to the invention accordingly permit selective effectiveness against chronic pain, preferably against neuropathic pain, more preferably against mononeuropathic/neuralgic or polyneuropathic pain, yet more preferably against pain in the case of post-herpetic neuralgia or in the case of diabetic polyneuropathy, preferably with negligible antinociceptive effectiveness in the case of acute pain. This unusual property of the compounds according to the invention is of fundamental importance for pain therapy as a whole.

A first aspect of the invention relates to compounds of the general formula (I)

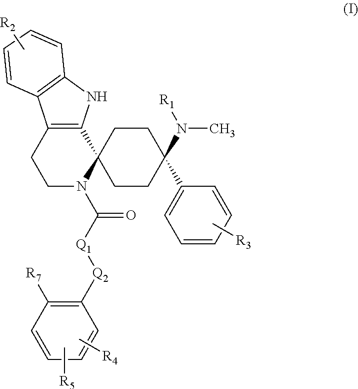

wherein
R₁ is —H or CH₃; preferably —CH₃;
R₂ is —H or -halogen; preferably —H or —F; particularly preferably —H;
R₃ is —H or -halogen; preferably -halogen; particularly preferably —F;
R₄ is —H, -halogen or —OC₁₋₃-alkyl; preferably —H or —OCH₃;
R₅ is —H, -halogen or —OC₁₋₃-alkyl; preferably —H or —OCH₃;
-Q₁-Q₂- forms the group —CH₂— or —CR₆═CH—; and
R₆ and R₇ are either both simultaneously —H or together via the bridge —S— form a five-membered ring;
   in the form of the free bases or physiologically acceptable salts.

When -Q₁-Q₂- forms the group —CH₂—, the compounds of the general formula (I) are phenylacetic acid amide derivatives.

When -Q₁-Q₂- forms the group —CR₆═CH—, the carbon atom of that group to which the radical R₆ is bonded is bonded to the carbon atom of the carbonyl group of the compound of the general formula (I). In that case, R₆ can be —H or ≠—H. When R₆ is —H, then R₇ is likewise —H. When R₆ is ≠—H, then R₆ and R₇ together form, via the bridge —S—, a five-membered ring, so that the compound of the general formula (I) is then a benzothiophene derivative.

The compounds according to the invention represent a selection from the compounds disclosed in U.S. Pat. No. 7,547,707 (=WO 2004/043967), U.S. Pat. No. 7,332,519 (=WO 2005/066183) and US 2008/0221141 (=WO 2006/108565). It has been found, surprisingly, that the spiroamines according to the invention which have the cis configuration on the cyclohexane ring in relation to the two nitrogens (cis-tetrahydro-spiro(cyclohexane-1,1′-pyrido[3,4-b]indole)-4-amine derivatives) have advantages over the other heterocycles.

Thus, the cis-spiroamides according to the invention, in contrast to the other compounds according to U.S. Pat. No. 7,547,707 (=WO 2004/043967), U.S. Pat. No. 7,332,519 (=WO 2005/066183) and US 2008/0221141 (=WO 2006/108565), exhibit in the animal model an outstanding action against chronic, preferably neuropathic, pain, more preferably pain in the case of diabetic polyneuropathy, without exhibiting a significant action against acute pain at the therapeutic dose required therefor. Because numerous side-effects of conventional analgesics are associated with the mechanism of action against acute pain, the spirocyclic cis-substituted cyclohexane derivatives according to the invention are distinguished by a particularly advantageous side-effect profile, in particular with regard to opioid-typical side-effects.

The compounds according to the invention are preferably achiral; the basic structure of the general formula (I) does not contain a chirality element (centre, axis or plane).

In relation to the spiro ring system, the compounds according to the invention are isomers, in which the substitution pattern on the spiro-cyclohexane ring system (not on the indole) can also be denoted cis/trans, Z/E or syn/anti. “Cis-trans isomers” are a subgroup of the stereoisomers (configuration isomers).

In the compounds according to the invention, the two nitrogen atoms of the spiroamine are in each case in the syn or cis or Z configuration relative to one another:

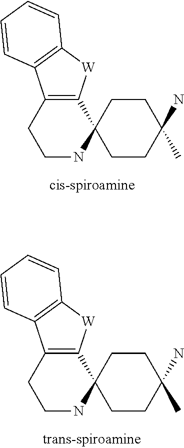

In one preferred embodiment, the excess of the cis-isomer so designated is at least 50% de, more preferably at least 75% de, yet more preferably at least 90% de, most preferably at least 95% de and in particular at least 99% de.

Suitable methods for separating the isomers (diastereoisomers) are known to persons skilled in the art. Examples which may be mentioned include column chromatography, preparative HPLC, and crystallization processes. Targeted synthesis processes, in which one isomer is formed in excess, are also known in principle to those skilled in the art.

The advantages of the cis-isomer are further particularly surprising in that, in the case of the structurally related spiroethers, it is usually not the cis-isomer but the trans-isomer that has properties which are advantageous from the pharmacological point of view (but which are occasionally of a different nature than the advantages of the cis-spiroamines according to the invention):

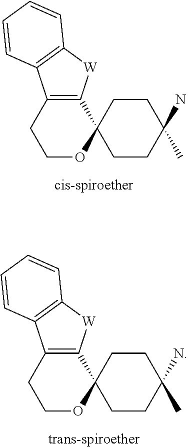

In one preferred embodiment, the compounds according to the invention are in the form of the free bases.

In another preferred embodiment, the compounds according to the invention are in the form of the physiologically acceptable salts.

For the purposes of the description, a “salt” is to be understood as being any form of the compound in which it assumes an ionic form or is charged and is coupled with a counter-ion (a cation or anion) or is in solution. The term is also to be understood as meaning complexes of the compound with other molecules and ions, in particular complexes which are associated via ionic interactions. Preferred salts are physiologically acceptable, in particular physiologically acceptable salts with anions or acids or also a salt formed with a physiologically acceptable acid.

Physiologically acceptable salts with anions or acids are salts of the particular compound in question with inorganic or organic acids which are physiologically acceptable—in particular when used in humans and/or mammals. Examples of physiologically acceptable salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, saccharinic acid, monomethylsebacic acid, 5-oxo-proline, hexane-1-sulfonic acid, nicotinic acid, 2-, 3- or 4-aminobenzoic acid, 2,4,6-trimethyl-benzoic acid, α-liponic acid, acetylglycine, acetylsalicylic acid, hippuric acid and/or aspartic acid. Particular preference is given to the hydrochloride, the citrate and the hemicitrate.

In one preferred embodiment, the compound according to the invention is in the form of the free compound or in the form of a physiologically acceptable salt, but preferably not in the form of a salt of benzenesulfonic acid, a salt of hydrochloric acid or a salt of citric acid.

For the purposes of this application, “-halogen” means preferably —F, —Cl, —Br or —I, more preferably —F or —Cl, in particular —F.

For the purposes of this application, “C₁₋₃-alkyl”, in each case independently, may be linear or branched, saturated or mono- or poly-unsaturated. Thus, “C₁₋₃-alkyl” includes acyclic saturated or unsaturated hydrocarbon radicals which can be branched or straight-chain, that is to say C₁₋₃-alkanyls, C₁₋₃-alkenyls and C₁₋₃-alkynyls.

Preferred forms of the compounds of the general formula (I) are compounds of the general formula (II), (III) or (IV):

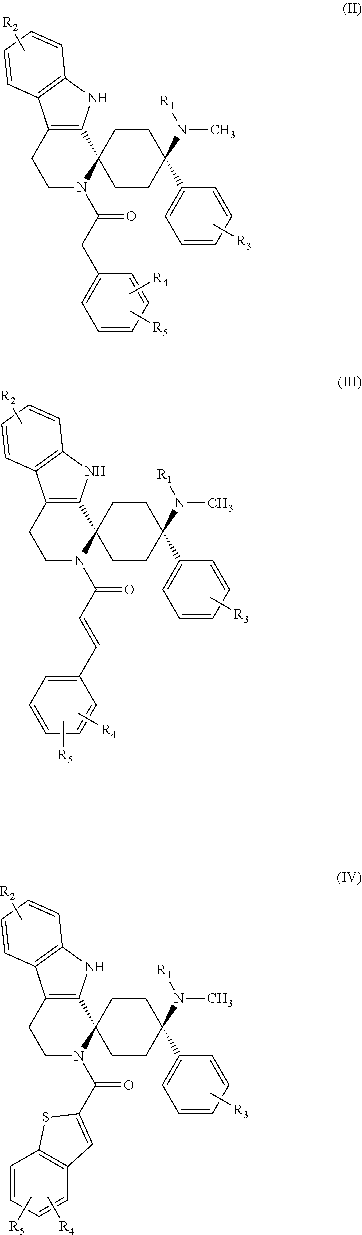

in the form of the free bases or physiologically acceptable salts.

Preferably, R₂ is —H and/or R₃ is —F.

Preferably, R₄ and R₅ are either both —H or both —OCH₃.

In a particularly preferred embodiment, the invention relates to compounds selected from the group consisting of compounds of the formulas (V), (VI) and (VII)

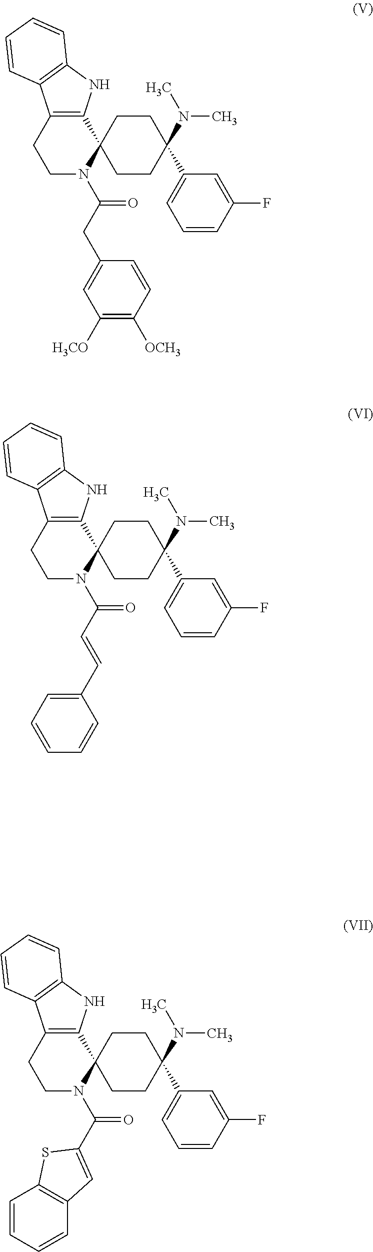 [SMILES: COc1ccc(CC(=O)N2CCc3c4ccccc4[nH]c3[C@]23CC[C@](c2cccc(F)c2)(N(C)C)CC3)cc1OC ; CN(C)[C@]1(c2cccc(F)c2)CC[C@]2(CC1)c1[nH]c3ccccc3c1CCN2C(=O)/C=C/c1ccccc1 ; CN(C)[C@]1(c2cccc(F)c2)CC[C@]2(CC1)c1[nH]c3ccccc3c1CCN2C(=O)c1cc2ccccc2s1]

in the form of the free bases or physiologically acceptable salts.

The free base of the compound of the general formula (V) can systematically be designated 2′,3′,4′,9′-tetrahydro-N,N-dimethyl-4-(3-fluorophenyl)-2′-(3,4-dimethoxy-benzyl)carbonyl-spiro[cyclohexane-1,1′(1′H)-pyrido[3,4-b]indole]-4-amine (cis-diastereoisomer) or also as 2-(3,4-dimethoxyphenyl)-1-((1s,4s)-4-(dimethylamino)-4-(3-fluorophenyl)-3,4′-dihydrospiro[cyclohexane-1,1′-pyrido[3,4-b]indol]-2′(9′H)-yl)ethanone. This compound is preferably in the form of the free base, in hydrochloride form, in citrate form or in hemicitrate form.

The free base of the compound of the general formula (VI) can systematically be designated (E)-2′,3′,4′,9′-tetrahydro-N,N-dimethyl-4-(3-fluorophenyl)-2′-(2-phenylvinyl)carbonyl-spiro[cyclohexane-1,1′(1′H)-pyrido[3,4-b]indole]-4-amine (cis-diastereoisomer) or also as (E)-1-((1s,4s)-4-(dimethylamino)-4-(3-fluorophenyl)-3′,4′-dihydrospiro[cyclohexane-1,1′-pyrido[3,4-b]indol]-2′(9′H)-yl)-3-phenylprop-2-en-1-one. This compound is preferably in the form of the free base, in hydrochloride form, in citrate form or in hemicitrate form.

The free base of the compound of the general formula (VII) can systematically be designated 2′,3′,4′,9′-tetrahydro-N,N-dimethyl-4-(3-fluorophenyl)-2′-(3,4-dimethoxybenzyl)carbonyl-spiro[cyclohexane-1,1′(1′H)-pyrido[3,4-b]indole]-4-amine (cis-diastereoisomer) or also as benzo[b]thiophen-2-yl((1 s,4s)-4-(dimethylamino)-4-(3-fluorophenyl)-3,4′-dihydrospiro[cyclohexane-1,1′-pyrido[3,4-b]indol]-2′(9′H)-yl)methanone. This compound is preferably in the form of the free base, in hydrochloride form, in citrate form or in hemicitrate form.

Compounds that are particularly preferred according to the invention are selected from the group consisting of:

| | |
|---|---|
| (E)-2′,3′,4′,9′-tetrahydro-N,N-dimethyl-4-phenyl-2′-(2- | AMD-1^{cis} |
| phenylvinyl)carbonyl-spiro[cyclohexane-1,1′(1′H)-pyrido[3,4-b]indole]-4- | |
| amine (cis-diastereoisomer) or (E)-1-((1s,4s)-4-(dimethylamino)-4- | |
| phenyl-3′,4′-dihydrospiro[cyclohexane-1,1′-pyrido[3,4-b]indol]-2′(9′H)-yl)- | |
| 3-phenylprop-2-en-1-one | |
| 2′,3′,4′,9′-tetrahydro-N,N-dimethyl-4-(3-fluoro-phenyl)-2′-(4- | AMD-2^{cis} |
| chlorobenzyl)-carbonyl-spiro[cyclohexane-1,1′(1′H)-pyrido[3,4-b]indole]- | |
| 4-amine (cis-diastereoisomer) or 2-(4-chlorophenyl)-1-((1s,4s)-4- | |
| (dimethylamino)-4-(3-fluorophenyl)-3′,4′-dihydrospiro[cyclohexane-1,1′- | |
| pyrido[3,4-b]indol]-2′(9′H)-yl)ethanone | |
| 2′,3′,4′,9′-tetrahydro-N,N-dimethyl-4-(3-fluorophenyl)-2′-(benzothiophen- | AMD-3^{cis} |
| 2-yl)carbonyl-spiro[cyclohexane-1,1′(1′H)-pyrido[3,4-b]indole]-4-amine | |
| (cis-diastereoisomer) or benzo[b]thiophen-2-yl((1s,4s)-4- | |
| (dimethylamino)-4-(3-fluorophenyl)-3′,4′-dihydrospiro[cyclohexane-1,1′- | |
| pyrido[3,4-b]indol]-2′(9′H)-yl)methanone | |
| 2′,3′,4′,9′-tetrahydro-N,N-dimethyl-4-(3-fluoro-phenyl)-2′-(4-fluorobenzyl)- | AMD-4^{cis} |
| carbonyl-spiro[cyclohexane-1,1′(1′H)-pyrido[3,4-b]indole]-4-amine (cis- | |
| diastereoisomer) or 1-((1s,4s)-4-(dimethylamino)-4-(3-fluorophenyl)- | |
| 3′,4′-dihydrospiro[cyclohexane-1,1′-pyrido[3,4-b]indol]-2′(9′H)-yl)-2-(4- | |
| fluorophenyl)ethanone | |
| (E)-2′,3′,4′,9′-tetrahydro-N,N-dimethyl-4-(3-fluorophenyl)-2′-(2- | AMD-5^{cis} |
| phenylvinyl)carbonyl-spiro[cyclohexane-1,1′(1′H)-pyrido[3,4-b]indole]-4- | AMD-6^{cis} |
| amine (cis-diastereoisomer) or (E)-1-((1s,4s)-4-(dimethylamino)-4-(3- | |
| fluorophenyl)-3′,4′-dihydrospiro[cyclohexane-1,1′-pyrido[3,4-b]indol]- | |
| 2′(9′H)-yl)-3-phenylprop-2-en-1-one | |
| 2′,3′,4′,9′-tetrahydro-N,N-dimethyl-4-(3-fluorophenyl)-2′-(3,4- | AMD-7^{cis} |
| dimethoxybenzyl)carbonyl-spiro[cyclohexane-1,1′(1′H)-pyrido[3,4- | |
| b]indole]-4-amine (cis-diastereoisomer) or 2-(3,4-dimethoxyphenyl)-1- | |
| ((1s,4s)-4-(dimethylamino)-4-(3-fluorophenyl)-3′,4′- | |
| dihydrospiro[cyclohexane-1,1′-pyrido[3,4-b]indol]-2′(9′H)-yl)ethanone | |
| (E)-2′,3′,4′,9′-tetrahydro-N,N-dimethyl-6′-fluoro-4-(3-fluorophenyl)-2′-(2- | AMD-8^{cis} |
| phenylvinyl)carbonyl-spiro[cyclohexane-1,1′(1′H)-pyrido[3,4-b]indole]-4- | |
| amine (cis-diastereoisomer) or (E)-1-((1s,4s)-4-(dimethylamino)-6′- | |
| fluoro-4-(3-fluorophenyl)-3′,4′-dihydrospiro[cyclohexane-1,1′-pyrido[3,4- | |
| b]indol]-2′(9′H)-yl)-3-phenylprop-2-en-1-one | |
| 2′,3′,4′,9′-tetrahydro-N,N-dimethyl-6′-fluoro-4-(3-fluorophenyl)-2′- | AMD-9^{cis} |
| (benzyl)carbonyl-spiro[cyclohexane-1,1′(1′H)-pyrido[3,4-b]indole]-4- | |
| amine (cis-diastereoisomer) or 1-((1s,4s)-4-(dimethylamino)-6′-fluoro-4- | |
| (3-fluorophenyl)-3′,4′-dihydrospiro[cyclohexane-1,1′-pyrido[3,4-b]indol]- | |
| 2′(9′H)-yl)-2-phenylethanone | |
| (E)-2′,3′,4′,9′-tetrahydro-N,N-dimethyl-6′-fluoro-4-phenyl-2′-(2- | AMD-10^{cis} |
| phenylvinyl)carbonyl-spiro[cyclohexane-1,1′(1′H)-pyrido[3,4-b]indole]-4- | |
| amine (cis-diastereoisomer) or (E)-1-((1s,4s)-4-(dimethylamino)-6′- | |
| fluoro-4-phenyl-3′,4′-dihydrospiro[cyclohexane-1,1′-pyrido[3,4-b]indol]- | |
| 2′(9′H)-yl)-3-phenylprop-2-en-1-one | |
| 2′,3′,4′,9′-tetrahydro-N,N-dimethyl-4-(3-fluorophenyl)-2′-benzylcarbonyl- | AMD-11^{cis} |
| spiro[cyclohexane-1,1′(1′H)-pyrido[3,4-b]indole]-4-amine (cis- | |
| diastereoisomer) or 1-((1s,4s)-4-(dimethylamino)-4-(3-fluorophenyl)- | |
| 3′,4′-dihydrospiro[cyclohexane-1,1′-pyrido[3,4-b]indol]-2′(9′H)-yl)-2- | |
| phenylethanone | |
| (E)-2′,3′,4′,9′-tetrahydro-N,N-dimethyl-4-(4-fluorophenyl)-2′-(2-phenyl- | AMD-12^{cis} |
| vinyl)carbonyl-spiro[cyclohexane-1,1′(1′H)-pyrido[3,4-b]indole]-4-amine | |
| (cis-diastereoisomer) or (E)-1-((1s,4s)-4-(dimethylamino)-4-(4- | |
| fluorophenyl)-3′,4′-dihydrospiro[cyclohexane-1,1′-pyrido[3,4-b]indol]- | |
| 2′(9′H)-yl)-3-phenylprop-2-en-1-one | |

and their physiologically acceptable salts and/or solvates, in particular the free bases, hydrochlorides, citrates or hemicitrates.

A further aspect of the invention relates to the compounds according to the invention as medicaments.

A further aspect of the invention relates to the compounds according to the invention for use in the treatment of neuropathic and/or chronic pain, administration preferably being twice daily, once daily or less frequently, particularly preferably not more than once daily.

The invention further provides the compounds according to the invention for use in the treatment of chronic pain. Preference is given to chronic pain selected from the group consisting of inflammatory pain, visceral pain, tumour pain and neuropathic pain. Neuropathic pain can be of mononeuropathic/neuralgic or polyneuropathic origin.

The invention further provides the compounds according to the invention for use in the treatment of pain in the case of diabetic polyneuropathy.

The invention further provides the compounds according to the invention for use in the treatment of pain as a result of post-herpetic neuralgia.

The compounds according to the invention are suitable for the treatment of neuropathic pain, preferably of mononeuropathic/neuralgic or polyneuropathic pain. The pain is preferably peripheral polyneuropathic pain or central polyneuropathic pain.

The polyneuropathy or the polyneuropathic pain is preferably acute (up to four weeks), subacute (from four to eight weeks) or chronic (more than eight weeks).

In the polyneuropathy, the motor, sensory, autonomic, sensorimotor or central nervous system is preferably affected. The symptoms are preferably distributed symmetrically or asymmetrically. The pain can be mild, moderate, medium-severe, severe or very severe. The neuropathic pain scale (NPS) can be used as a measure (see B. S. Galer et al., Neurology 1997, 48, 332-8).

Examples of causes of peripheral neuropathic pain are diabetic polyneuropathy, post-herpetic neuralgia, radioculopathy, post-traumatic neuralgia, polyneuropathy induced by chemical substances, for example by chemotherapy, phantom pain of the limbs, complex regional syndrome, HIV-induced sensory polyneuropathy and alcoholic polyneuropathy. Examples of causes of central polyneuropathic pain are compressive myelopathy as a result of narrowed canal stenosis, post-traumatic spinal pain, pain due to stroke, post-ischaemic myelopathy, radiation-induced myelopathy, myelopathy induced by multiple sclerosis, and HIV-induced myelopathy.

In one preferred embodiment, the neuropathy causing the neuropathic pain is associated with a disease selected from the group consisting of Diabetes mellitus, vasculitis, uraemia, hypothyroidism, alcohol abuse, post-herpetic neuralgia, idiopathic neuropathy, chronic inflammatory demyelinating neuropathy, multifocal motor neuropathy, hereditary polyneuropathy, Guillain-Barré syndrome, intoxication [e.g. by alcohol, heavy metals {in particular Pb, Hg, As}, hydrocarbons, as a result of chemotherapy with cytostatics], porphyria, infectious diseases, cancer diseases [e.g. myeloma, amyloid, leukaemia, lymphoma], pernicious anaemia, vitamin E deficiency, Refsum's disease, Bassen-Kornzweig syndrome, Fabry's disease, vasculitis and amyloidosis. Diabetic polyneuropathy and post-herpetic neuralgia are particularly preferred. If the disease is an infectious disease, it is preferably selected from the group consisting of mononucleosis, ehrlichiosis, typhus, diphtheria, leprosy, HIV, lues and borreliosis.

The polyneuropathic pain is preferably pain caused by a polyneuropathy within the meaning of the ICD-10 (International Statistical Classification of Diseases and Related Health Problems, WHO Edition, preferably as at 2008).

The invention further provides the compounds according to the invention for use in the treatment of anxiety states, stress and stress-associated syndromes, depression, epilepsy, Alzheimer's disease, senile dementia, general cognitive dysfunctions, learning and memory disorders (as a nootropic), withdrawal symptoms, alcohol and/or drug and/or medicament abuse and/or dependency, sexual dysfunctions, cardiovascular diseases, hypotension, hypertension, tinnitus, pruritus, migraine, hardness of hearing, insufficient intestinal motility, impaired food intake, anorexia, obesity, locomotor disorders, diarrhoea, cachexia, urinary incontinence, or as a muscle relaxant, anticonvulsive or anaesthetic, or for coadministration in the case of treatment with an opioid analgesic or with an anaesthetic, for diuresis or antinatriuresis, anxiolysis, for modulation of locomotor activity, for modulation of neurotransmitter excretion and treatment of neurodegenerative diseases associated therewith, for the treatment of withdrawal symptoms and/or to reduce the addictive potential of opioids.

The invention further provides a method of treating, in particular in one of the above-mentioned indications, a non-human mammal or a human requiring treatment of chronic pain, preferably neuropathic pain, more preferably pain in the case of diabetic polyneuropathy or post-herpetic neuralgia, by administering an individually therapeutically necessary daily dose of a compound according to the invention, or of a form of administration according to the invention, whereby there is at the same time preferably no significant suppression of the sensation of acute nociceptor pain and/or no occurrence of significant opioid-typical side-effects, in particular there is substantially no respiratory depression and/or constipation and/or urinary retention and/or nausea and/or vomiting and/or hypotonia and/or bradycardia and/or addiction and/or dependency and/or euphoria and/or depression and/or sedation and/or dizziness.

The invention further provides a method of treating, in particular in one of the above-mentioned indications, a non-human mammal or human requiring treatment of chronic pain, preferably neuropathic pain, more preferably pain in the case of diabetic polyneuropathy or post-herpetic neuralgia, by administering a daily dose X of a compound according to the invention, or of a form of administration according to the invention, whereby there is preferably no significant simultaneous suppression of the sensation of acute nociceptor pain and/or no occurrence of significant opioid-typical side-effects, in particular there is substantially no respiratory depression and/or constipation and/or urinary retention and/or nausea and/or vomiting and/or hypotonia and/or bradycardia and/or addiction and/or dependency and/or euphoria and/or depression and/or sedation and/or dizziness; wherein the daily dose X is selected from the group consisting of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10 mg.

The invention further provides compounds according to the invention having affinity for the μ-opioid receptor and for the ORL-1 receptor, which
are significantly effective in the treatment of neuropathic pain, preferably in the rat, more preferably as mononeuropathic pain in the model according to Chung, and are characterised by a half-maximum effective dose ED₅₀ⁿ, and
are substantially not significantly effective in the treatment of acute pain, preferably in the rat, more preferably in the tail-flick test, in a dose which is higher than ED₅₀ⁿ by a factor of 5.

Accordingly, the compounds according to the invention, when administered in that half-maximum effective dose ED₅₀ⁿ, which is defined in relation to the effectiveness of the compound against neuropathic pain, and even in a dose that is higher than ED₅₀ⁿ by a factor of 5, exhibit—if at all—at most a negligible antinociceptive action in the case of acute pain, preferably in the rat, more preferably in the tail-flick test.

In a preferred embodiment, the neuropathic pain is mononeuropathic or neuralgic pain, preferably pain as a result of post-herpetic neuralgia. In another preferred embodiment, the pain is polyneuropathic pain, preferably pain in the case of diabetic polyneuropathy.

Preferably, the compounds according to the invention are substantially not significantly effective in the treatment of acute or nociceptive pain even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200, 300, 400 or 500, most preferably by a factor of 600, 700, 800 or 900, and in particular by a factor of 1000.

The half-maximum effective dose ED₅₀ⁿ is known to the person skilled in the art. It is preferably defined as the dose at which, with regard to the treatment of neuropathic pain, 50% of the maximum therapeutic action is achieved. Accordingly, a half-maximum effective dose ED₅₀^{a} can be defined as the dose at which, with regard to the treatment of acute pain, 50% of the maximum therapeutic action is achieved. The compounds according to the invention are defined by ED₅₀ⁿ, however, not by ED₅₀^{a}.

Suitable methods for studying the effectiveness of an active ingredient in the treatment of neuropathic pain and for determining the half-maximum effective dose ED₅₀ⁿ in the treatment of neuropathic pain are known to the person skilled in the art. The same is true for studying the effectiveness of an active ingredient against acute pain.

For example, the determination can be carried out in an animal model (e.g. mouse or rat), whereby
mononeuropathic pain can be studied according to Chung (S. H. Kim, J. M. Chung, Pain. 1992, 50(3), 355-63) or Bennett (G. J. Bennett, Y. K. Xie, Pain. 1988, 33(1), 87-107),
pain in the case of diabetic polyneuropathy can be studied by streptozotocin (STZ)-induced diabetes (E. K. Joseph, J. D. Levine, Neuroscience. 2003; 120(4):907-13), and
acute pain can be studied in the so-called tail-flick test (D'Amour and Smith, J. Pharm. Exp. Ther. 72, 1941, 74-9).

The determination is preferably carried out in the animal model, with regard to the effectiveness against neuropathic pain as effectiveness against mononeuropathic pain in the rat in the model according to Chung, and with regard to the effectiveness against acute pain in the rat in the tail-flick test, preferably in each case as described in the experimental section.

Accordingly, the compounds according to the invention preferably have an affinity for the μ-opioid receptor and for the ORL-1 receptor which, in the rat,
are significantly effective in the treatment of mononeuropathic pain in the model according to Chung and are characterised by a half-maximum effective dose ED₅₀ⁿ, and
are not significantly effective in the treatment of acute pain in the tail-flick test in a dose which is higher than ED₅₀ⁿ by a factor of 5.

The evaluation of the experimental findings in respect of statistically significant differences between the dose groups and the vehicle-control groups is preferably carried out by means of variance analysis with repeated measures (repeated measures ANOVA) and a post hoc analysis according to Bonferroni, preferably as described in the experimental section. The significance level is set at p<0.05. The group sizes are usually n=10.

In principle, the comparative determination of analgesic effectiveness against neuropathic pain and acute, nociceptive pain can also be carried out in humans, but this is less preferred inter alia for ethical reasons. The study of effectiveness against neuropathic pain, that is to say in patients suffering from neuropathic pain, can then be carried out according to Hansson P, Backonja M, Bouhassira D. (2007). Usefulness and limitations of quantitative sensory testing: clinical and research application in neuropathic pain states. Pain. 129(3): 256-9. The study of effectiveness against acute pain can then be carried out according to Posner J, Telekes A, Crowley D, Phillipson R, Peck A W. (1985). Effects of an opiate on cold-induced pain and the CNS in healthy volunteers. Pain. 23(1):73-82.

It has been found, surprisingly, that the compounds according to the invention are distinguished by a very advantageous side-effects profile as compared with conventional stage-3 opioids. Thus, even on administration of therapeutically effective doses, as are required in particular for the treatment of neuropathic pain, no or at most only slightly pronounced opioid-typical side-effects are observed, such as, for example, respiratory depression, constipation, urinary retention, nausea, vomiting, hypotonia, bradycardia, addiction, dependency, euphoria, depression, sedation and dizziness. Hitherto, the greatly reduced occurrence of the opioid-typical side-effects respiratory depression, constipation, hypotonia, bradycardia, disturbance of motor coordination capacity (as a measure of central-nervous side-effects), physical and mental dependency has been shown experimentally in animal models.

In a preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant respiratory depression as a side-effect, preferably in the rat, more preferably in the blood gas analysis model. Preferably, the compounds according to the invention do not exhibit significant respiratory depression as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200.

Suitable methods for studying active-ingredient-induced respiratory depression are known to the person skilled in the art. The study is preferably carried out in a blood gas analysis model in the rat as the change in the arterial O₂ and CO₂ partial pressures. Evaluation of the experimental findings in respect of statistically significant differences between the dose groups and the vehicle-control groups is preferably carried out by means of single-factor variance analysis (one-way ANOVA) as well as a post hoc analysis according to Dunnett, preferably as described in the experimental section. The significance level is set at p<0.05. The group sizes are usually n=6. For further details of this animal model, reference is also made to the experimental section.

In one preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant constipation as a side-effect, preferably in the mouse, more preferably in the charcoal passage test. Preferably, the compounds according to the invention do not exhibit significant constipation as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200, 300, 400 or 500, most preferably by a factor of 600.

Suitable methods for studying active-ingredient-induced constipation are known to the person skilled in the art. The study is preferably carried out in a charcoal passage model in the mouse as the change in the gastrointestinal transit speed. Evaluation of the experimental findings in respect of statistically significant differences between the dose groups and the vehicle-control groups is preferably carried out by means of single-factor variance analysis (one-way ANOVA) as well as a post hoc analysis according to Dunnett, preferably as described in the experimental section. The significance level is set at p<0.05. The group sizes are usually n=10. For further details of this animal model, reference is also made to the experimental section.

In one preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant hypotonia as a side-effect, preferably in awake rabbits, more preferably in the circulatory model in awake rabbits with telemetry. Preferably, the compounds according to the invention do not exhibit significant hypotonia as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200.

Suitable methods for studying active-ingredient-induced hypotonia are known to the person skilled in the art. The study is preferably carried out in a circulatory model in awake rabbits with telemetry as the change in the arterial blood pressure (systolic, diastolic and mean value). Evaluation of the experimental findings in respect of statistically significant differences between the dose groups and the vehicle-control groups is preferably carried out by means of single-factor variance analysis (one-way ANOVA) as well as a post hoc analysis according to Dunnett, preferably as described in the experimental section. The significance level is set at p<0.05. The group sizes are usually n=6. For further details of this animal model, reference is also made to the experimental section.

In a preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant bradycardia as a side-effect, preferably in awake rabbits, more preferably in the circulatory model in awake rabbits with telemetry. Preferably, the compounds according to the invention do not exhibit significant bradycardia as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200.

Suitable methods for studying active-ingredient-induced bradycardia are known to persons skilled in the art. The study is preferably carried out in a circulatory model in awake rabbits with telemetry as the change in the cardiac frequency. Evaluation of the experimental findings in respect of statistically significant differences between the dose groups and the vehicle-control groups is preferably carried out by means of single-factor variance analysis (one-way ANOVA) as well as a post hoc analysis according to Dunnett, preferably as described in the experimental section. The significance level is set at p<0.05. The group sizes are usually n=6. For further details of this animal model, reference is also made to the experimental section.

In one preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant disturbance of motor coordination capacity (as a measure of central-nervous side-effects) as a side-effect, preferably in the mouse, more preferably in the RotaRod test. Preferably, the compounds according to the invention do not exhibit a significant disturbance of motor coordination capacity (as a measure of central-nervous side-effects) as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200, 300, 400 or 500, most preferably by a factor of 600, 700, 800 or 900, and in particular by a factor of 1000.

Suitable methods for studying an active-ingredient-induced disturbance of motor coordination capacity are known to persons skilled in the art. The study is preferably carried out in a RotaRod model in the mouse (analogously to Kuribara H., Higuchi Y., Tadokoro S. (1977), Effects of central depressants on Rota-Rod and traction performance in mice. Japan. J. Pharmacol. 27, 117-126) as the change in the ability to run on a rotating rod. Evaluation of the experimental findings in respect of statistically significant differences between the dose groups and the vehicle-control groups is preferably carried out by means of single-factor variance analysis (one-way ANOVA) as well as a post hoc analysis according to Dunnett, preferably as described in the experimental section. The significance level is set at p<0.05. The group sizes are usually n=10. For further details of this animal model, reference is also made to the experimental section.

In one preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant physical dependency or withdrawal symptoms as a side-effect, preferably in the mouse, more preferably in the jumping test. Preferably, the compounds according to the invention do not exhibit significant physical dependency or withdrawal symptoms as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200, 300, 400 or 500, most preferably by a factor of 600, 700, 800 or 900, and in particular by a factor of 1000.

Suitable methods for studying active-ingredient-induced physical dependency are known to persons skilled in the art. The study is preferably carried out in the jumping model in the mouse (analogously to Saelens J K, Arch Int Pharmacodyn 190: 213-218, 1971) as naloxone-induced withdrawal. Evaluation of the experimental findings in respect of statistically significant differences between the dose groups and the vehicle-control groups is preferably carried out by means of Fisher's exact test for the parameter “number of animals with withdrawal symptoms” as well as by means of the Kruskal-Wallis test for the parameter “jumping frequency”, preferably as described in the experimental section. The significance level is set at p<0.05 in each case. The group sizes are usually n=12. For further details of this animal model, reference is also made to the experimental section.

In one preferred embodiment, the compounds according to the invention, when administered in the half-maximum effective dose ED₅₀ⁿ, which is defined with regard to the effectiveness of the compound against neuropathic pain, and preferably even in a dose which is higher than ED₅₀ⁿ by a factor of 5, do not exhibit significant mental dependency or addiction as a side-effect, preferably in the rat, more preferably by means of conditioned place preference. Preferably, the compounds according to the invention do not exhibit significant mental dependency or addiction as a side-effect even in a dose which is higher than the half-maximum effective dose ED₅₀ⁿ by a factor of 10, 20, 30, 40 or 50, more preferably by a factor of 75, 100, 125, 150 or 175, yet more preferably by a factor of 200, 300, 400 or 500, most preferably by a factor of 600, 700, 800 or 900, and in particular by a factor of 1000.

Suitable methods for studying active-ingredient-induced mental dependency or addiction are known to persons skilled in the art. The study is preferably carried out by means of conditioned place preference in rats, preferably as described in Tzschentke, T. M., Bruckmann, W. and Friderichs, F. (2002) Lack of sensitization during place conditioning in rats is consistent with the low abuse potential of tramadol. Neuroscience Letters 329, 25-28. Evaluation of the experimental findings in respect of statistically significant differences in the animals' preference for the active ingredient or the vehicle is preferably carried out by means of a paired t-test. The significance level is set at p<0.05. The group sizes are usually n=8. For further details of this animal model, reference is made to the description of the method in Tzschentke, T. M., Bruckmann, W. and Friderichs, F. (2002) Neuroscience Letters 329, 25-28.

The compounds according to the invention are suitable for the treatment of chronic pain, preferably neuropathic pain, more preferably mononeuropathic/neuralgic or polyneuropathic pain, yet more preferably pain in the case of post-herpetic neuralgia or in the case of diabetic polyneuropathy.

The definitions of the different forms of chronic pain are known to the person skilled in the art. Reference may be made in this connection to, for example, Merskey H., Bogduk N. Classification of chronic pain. Seattle: IASP Press 1994, Bennett G. J., Anesth Analg. 2003, 97, 619-20 and Backonja M. M., Anesth Analg. 2003, 97, 785-90.

For the purposes of this application, chronic pain is preferably defined as pain which exists over a prolonged period (usually at least 3, 4, 5 or 6 months) and persists beyond the normal healing time. Neuropathic pain is preferably defined as pain or a sensory phenomenon which is caused by lesion, disease or dysfunction of the central or peripheral nervous system. For the purposes of the description, acute pain is preferably defined as an unpleasant sensory and emotional experience which accompanies acute or potential tissue damage or is described in the terms of such damage (see definition of the International Association for the Study of Pain® (IASP)).

The compounds according to the invention have a Kᵢ value on the μ-opioid receptor of preferably not more than 1000 nM, more preferably not more than 500 nM, yet more preferably 100 nM, most preferably not more than 50 nM and in particular not more than 25 nM.

Methods of determining the Kᵢ value on the μ-opioid receptor are known to persons skilled in the art. The determination is preferably carried out in a homogeneous batch in microtitre plates. To that end, serial dilutions of the substances to be tested are preferably incubated for 90 minutes at room temperature with a receptor membrane preparation (15-40 μg of protein per 250 μl of incubation batch) of CHO-K1 cells which express the human μ-opiate receptor (RB-HOM receptor membrane preparation from NEN, Zaventem, Belgium) in the presence of 1 nmol/l of the radioactive ligand [³H]-naloxone (NET719, NEN, Zaventem, Belgium) and 1 mg of WGA-SPA beads (wheat germ agglutinin SPA beads from Amersham/Pharmacia, Freiburg, Germany), in a total volume of 250 There is preferably used as the incubation buffer 50 mmol/l of Tris-HCl supplemented with 0.05 wt. % sodium azide and 0.06 wt. % bovine serum albumin. For the determination of non-specific binding, 25 μmol/l of naloxone are preferably added in addition. When the 90-minute incubation time is complete, the microtitre plates are preferably centrifuged off for 20 minutes at 1000 g and the radioactivity is measured in a β counter (Microbeta-Trilux, PerkinElmer Wallac, Freiburg, Germany). The percentage displacement of the radioactive ligand from its binding to the human μ-opiate receptor at a concentration of the test substances of preferably 1 μmol/l is determined and indicated as the percentage inhibition (% inhibition) of specific binding. On the basis of the percentage displacement by different concentrations of the compounds to be tested it is possible to calculate IC₅₀ inhibitory concentrations, which effect 50% displacement of the radioactive ligand. The Kᵢ values for the test substances can be calculated therefrom by conversion by means of the Cheng-Prusoff equation.

The compounds according to the invention have a Kᵢ value on the ORL1 receptor of preferably not more than 500 nM, more preferably not more than 100 nM, most preferably not more than 50 nM and in particular not more than 10 nM.

Methods for determining the Kᵢ value on the ORL1 receptor are known to persons skilled in the art. The determination is preferably carried out in a receptor binding assay with ³H-nociceptin/orphanin FQ with membranes of recombinant CHO-ORL1 cells. This test system is preferably carried out according to the method put forward by Ardati et al. (Mol. Pharmacol., 51, 1997, p. 816-824). The concentration of ³H-nociceptin/orphanin FQ in these tests is preferably 0.5 nM. The binding assays are preferably carried out with in each case 20 μg of membrane protein per 200 μl batch in 50 mM Hepes, pH 7.4, 10 mM MgCl₂ and 1 mM EDTA. Binding to the ORL1 receptor is preferably determined using in each case 1 mg of WGA-SPA beads (Amersham-Pharmacia, Freiburg) by incubating the batch for one hour at RT and then measuring in a Trilux scintillation counter (Wallac, Finland).

The invention further provides a process for the preparation of the compounds according to the invention. Suitable processes for the synthesis of the compounds according to the invention are known in principle to the person skilled in the art.

Preferred synthesis routes are described below:

### Synthesis of the Ketone Structural Units E:

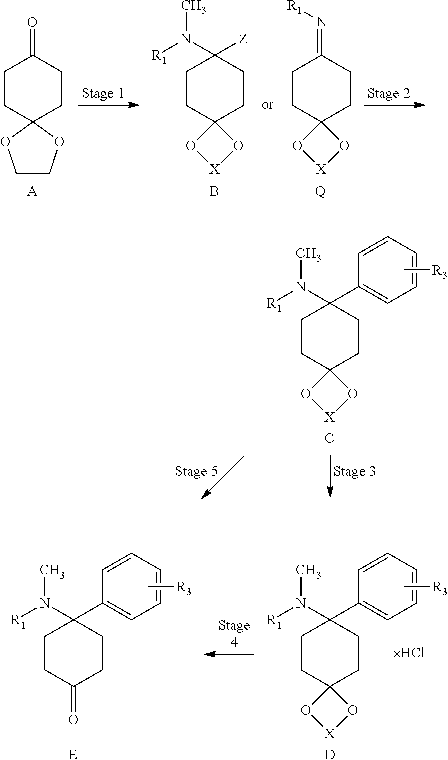

### Stage 1 (via 8)

Structures of formula B can be prepared by reaction of ketones A with amines and acidic reactants Z—H. Suitable reactants Z—H are, for example, hydrogen cyanide, 1,2,3-triazole, benzotriazole or pyrazole. A particularly preferred route to compounds of structure B is the reaction of ketones with metal cyanides and the corresponding amine in the presence of acid, preferably in an alcohol, at temperatures of from −40 to 60° C., preferably at room temperature with alkali metal cyanides in methanol. A further particularly preferred route to compounds of structure B is the reaction of ketones with 1,2,3-triazole and the corresponding amine in the presence under water-removing conditions, preferably using a water separator at elevated temperature in an inert solvent or using molecular sieve or another drying agent. In an analogous manner, structures analogous to B can be introduced using benzotriazole or pyrazole groups instead of triazole groups.

### Stage 1 (via Q)

The preparation of imines of the general formula Q from ketones A is to be found in the general prior art.

### Stage 2 (via 8)

In general, acetals C can be obtained by substitution of suitable leaving groups Z in structures of formula B. Suitable leaving groups are preferably cyano groups; 1,2,3-triazol-1-yl groups. Further suitable leaving groups are 1H-benzo[d][1,2,3]triazol-1-yl groups and pyrazol-1-yl groups (Katritzky et al., Synthesis 1989, 66-69). A particularly preferred route to compounds of structure C is the reaction of aminonitriles B (Z═CN) with corresponding organometallic compounds, preferably Grignard compounds, preferably in ethers, preferably at RT. The organometallic compounds are either available commercially or can be prepared according to the general prior art. A further particularly preferred route to compounds of structure C is the reaction of aminotriazoles B (Z=triazole) with corresponding organometallic compounds, preferably Grignard compounds, preferably in ethers, preferably at RT. The organometallic compounds are either available commercially or can be prepared according to the general prior art.

### Stage 2 (via Q)

Aminoacetals C having not more than one substituent on the nitrogen atom can be obtained according to processes known in principle to the person skilled in the art by addition of carbon nucleophiles to imines Q, preferably organometallic compounds in inert solvents, particularly preferably with Grignard reagents or organolithium compounds, preferably in ethers, preferably at temperatures of from 100 to RT.

### Stage 4/5:

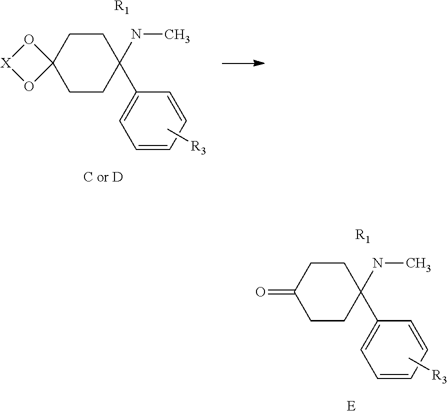

Compounds of formula E can be freed from corresponding acetals C, or from their salts D, according to generally known prior art by deprotection by means of acids. X is selected from the group alkyl, alkyl/alkylidene/alkylidene substituted by aryl or by alkyl (saturated/unsaturated).

Preparation of C(R₁≠—H) from Ca (R₁═—H)

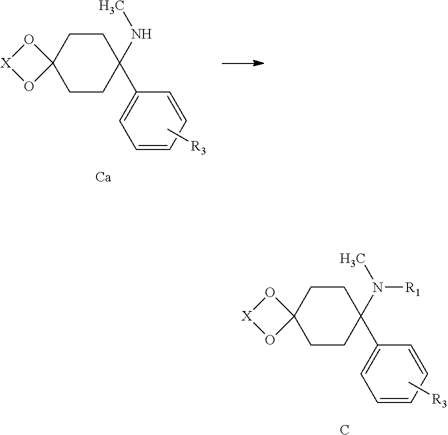

Aminoacetals Ca having not more than one substituent on the nitrogen atom can be converted according to processes known in principle to the person skilled in the art, for example by reductive amination, into corresponding aminoacetals C having one or two further substituents on the nitrogen.

### Aminonitrile Route, Imine Route and Triazole Route

The required ketone intermediates E can be prepared, for example, according to the following three different routes: (1) aminonitrile route, (2) imine route and (3) triazole route.

### (1) Aminonitrile Route:

In the aminonitrile route there is synthesised, as described in the following synthesis scheme, from a ketone precursor A the aminonitrile Ba, which is converted into the structural units C or D and further into E using a nucleophile MR3. This synthesis route has already been described and used in U.S. Pat. No. 7,547,707 (=WO 2004/043967), the entire disclosure of which is incorporated herein by reference.

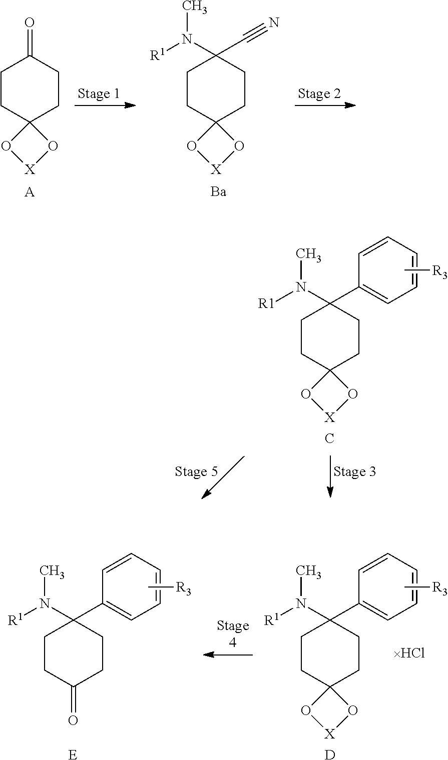

### (2) Imine Route:

In the imine route there is synthesised, as described in the following scheme, from a ketone precursor A the imine Q, which is converted into the structural units C and D and further into E using a nucleophile MR3. The required imine structural units Q can be prepared according to a method known to the person skilled in the art (Layer, Chem. Rev., 1963, 8, 489-510). For addition of the organometallic species MR3 to the imine Q, processes known in the literature (e.g. Maddox et al., J. Med. Chem., 1965, 8, 230-235. Kudzma et al., J. Med. Chem., 1989, 32, 2534-2542) were used. Stages 3, 4 and 5 are carried out analogously to the aminonitrile route.

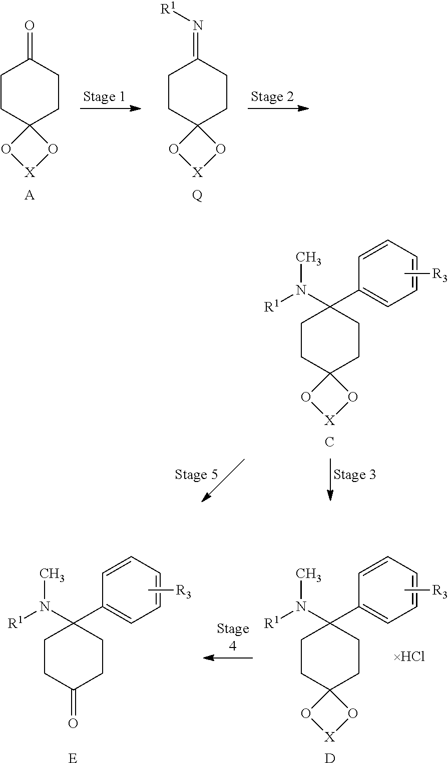

### (3) Triazole Route:

In the triazole route there was synthesised, as described in the following scheme, from a ketone precursor A the triazole Bb, which is converted into the structural units C and D and further into E using a nucleophile MR3. The conditions can be found in the indicated literature references: (a) Katritzky et al. Synthesis, 1992, 1295-1298. (b) Prashad, et al., Tetrahedron Lett. 2005, 46, 5455-5458.

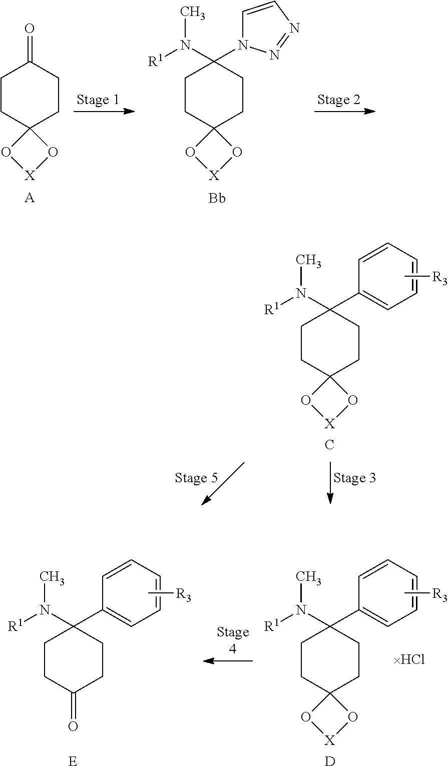

### Synthesis of the Spiroamines (AMN)

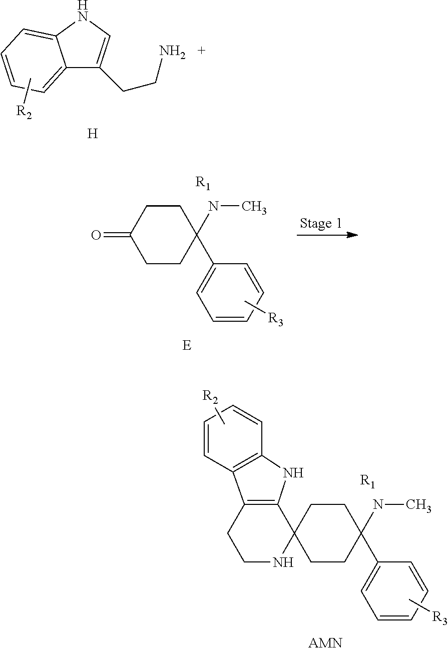

Tryptamines of type H can be reacted in reactions of the Pictet-Spengler reaction type with ketones E, with the addition of at least one reagent from the group of the acids, acid anhydrides, esters, weakly acid-reacting salts or Lewis acids, to form products of the formula AMN.

There is preferably used at least one reagent from the group carboxylic acids, phosphoric acids or sulfonic acids or their anhydrides, carboxylic acid trialkylsilyl esters, acid-reacting salts, mineral acids or Lewis acids selected from the group consisting of boron trifluoride, indium(III) chloride, titanium tetrachloride, aluminium(III) chloride, or with the addition of at least one transition metal salt, preferably with the addition of at least one transition metal triflate (transition metal trifluoromethanesulfonate), particularly preferably with the addition of at least one transition metal trifluoromethanesulfonate selected from the group consisting of scandium(III) trifluoromethanesulfonate, ytterbium(III) trifluoromethanesulfonate and indium(III) trifluoromethanesulfonate, optionally with the addition of Celite, with solid-phase-bound reactants or reagents, at elevated or reduced temperature, with or without microwave radiation, optionally in a suitable solvent or solvent mixture such as, for example, chlorinated or unchlorinated, then preferably aromatic, hydrocarbons, acetonitrile; in ethereal solvents, preferably in diethyl ether or THF; or in nitromethane, in suitable cases also in alcohols or water. Particular preference is given to the use of pyridinium para-toluenesulfonate, phosphorus pentoxide in the presence of Celite, boron trifluoride etherate, trifluoroacetic acid, ortho-titanic acid tetraisopropyl ester together with trifluoroacetic acid, trifluoromethanesulfonic acid trimethylsilyl ester, trifluoromethanesulfonic acid, methanesulfonic acid, trifluoroacetic acid, acetic acid, phosphoric acid, polyphosphoric acid, polyphosphate esters, p-toluenesulfonic acid, hydrochloric acid HCl gas, sulfuric acid together with acetate buffer, tin tetrachloride.

The conditions indicated in the following examples are in turn preferably used.

Compounds of the general formulae H and E are either available commercially or their preparation is known from the prior art or can be derived from the prior art in a manner obvious to the person skilled in the art. The following citations are particularly relevant in this connection: Jirkovsky et al., J. Heterocycl. Chem., 12, 1975, 937-940; Beck et al., J. Chem. Soc. Perkin 1, 1992, 813-822; Shinada et al., Tetrahedron Lett., 39, 1996, 7099-7102; Garden et al., Tetrahedron, 58, 2002, 8399-8412; Lednicer et al., J. Med. Chem., 23, 1980, 424-430; Bandini et al. J. Org. Chem. 67, 15; 2002, 5386-5389; Davis et al., J. Med. Chem. 35, 1, 1992, 177-184; Yamagishi et al., J. Med. Chem. 35, 11, 1992, 2085-2094; Gleave et al.; Bioorg. Med. Chem. Lett. 8, 10, 1998, 1231-1236; Sandmeyer, Helv. Chim. Acta; 2; 1919; 239; Katz et al.; J. Med. Chem. 31, 6, 1988; 1244-1250; Bac et al. Tetrahedron Lett. 1988, 29, 2819; Ma et al. J. Org. Chem. 2001, 66, 4525; Kato et al. J. Fluorine Chem. 99, 1, 1999, 5-8.

Synthesis of the spiroamides (AMD)

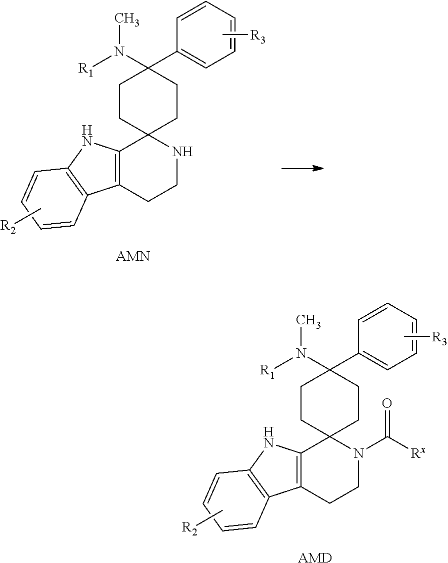

Compounds of the general formula AMN can be reacted with carboxylic acids in at least one solvent, preferably selected from the group consisting of dichloromethane, acetonitrile, dimethylformamide, diethyl ether, dioxane and tetrahydrofuran, with the addition of at least one coupling reagent, preferably selected from the group consisting of carbonyldiimidazole (CU), 2-chloro-1-methylpyridinium iodide (Mukaiyama reagent), N-(3-dimethylaminopropyl)-N′-ethylcarbodiimide (EDCI), O-(benzotriazol-1-yl)-N,N,N′,N′-tetramethyluronium tetrafluoroborate (TBTU), N,N′-dicyclohexylcarbodiimide (DCC) and 1-benzotriazolyloxy-tris-(dimethylamino)-phosphonium hexafluorophosphate (BOP), optionally in the presence of at least one inorganic base, preferably selected from the group consisting of potassium carbonate and caesium carbonate, or of an organic base, preferably selected from the group consisting of triethylamine, diisopropylethylamine and pyridine, and optionally with the addition of 4-(dimethylamino)pyridine or 1-hydroxybenzotriazole, at temperatures of preferably from 25° C. to 150° C., optionally with microwave radiation, to give compounds of the general formula AMD.

Compounds of the general formula AMN can be reacted with acid anhydrides and carboxylic acid chlorides in at least one solvent, preferably selected from the group consisting of dichloromethane, acetonitrile, dimethylformamide, diethyl ether, dioxane and tetrahydrofuran, optionally in the presence of at least one inorganic base, preferably selected from the group consisting of potassium carbonate and caesium carbonate, or of an organic base, preferably selected from the group consisting of triethylamine, diisopropylethylamine and pyridine, and optionally with the addition of 4-(dimethylamino)pyridine or 1-hydroxybenzotriazole, at temperatures of preferably from 25° C. to 150° C., optionally with microwave radiation, to give compounds of the general formula AMD.

Further details regarding the synthesis of compounds according to the invention, particularly regarding the synthesis of suitable starting structural units, are described in U.S. Pat. No. 7,547,707 (=WO 2004/043967), U.S. Pat. No. 7,288,560 (=WO 2005/063769), U.S. Pat. No. 7,332,519 (=WO 2005/066183), U.S. Pat. No. 7,776,848 (=WO 2006/018184), US 2008/0221141 (=WO 2006/108565), US 2009/0111842 (=WO 2007/124903) and US 2009/0156593 (=WO 2008/009416), the entire disclosure of each of which is incorporated herein by reference. A person skilled in the art will recognize that suitable starting structural units for the synthesis of the compounds according to the invention can be prepared analogously to the synthesis schemes and implementation examples disclosed in these publications.

The compounds according to the invention act, for example, on the ORL1 and μ-opioid receptors, which are relevant in connection with various diseases, so that they are suitable as an active ingredient (medicament) in a pharmaceutical composition.

The invention further provides a pharmaceutical composition which contains a physiologically acceptable carrier and at least one compound according to the invention.

Preferably, the composition according to the invention
is solid, liquid or pasty; and/or
contains the compound according to the invention in an amount of from 0.001 to 99 wt. %, preferably from 1.0 to 70 wt. %, based on the total weight of the composition.

The pharmaceutical composition according to the invention can optionally contain suitable additives and/or auxiliary substances and/or optionally further active ingredients.

Examples of suitable physiologically acceptable carriers, additives and/or auxiliary substances are fillers, solvents, diluents, colourings and/or binders. These substances are known to the person skilled in the art (see H. P. Fiedler, Lexikon der Hilfsstoffe für Pharmazie, Kosmetik and angrenzende Gebiete, Editio Cantor Aulendoff).

The composition according to the invention contains the compound according to the invention in an amount of preferably from 0.001 to 99 wt. %, more preferably from 0.1 to 90 wt. %, yet more preferably from 0.5 to 80 wt. %, most preferably from 1.0 to 70 wt. % and in particular from 2.5 to 60 wt. %, based on the total weight of the composition.

The composition according to the invention is preferably produced for systemic, topical or local administration, preferably for oral administration.

The invention further provides a pharmaceutical form of administration which contains the pharmaceutical composition according to the invention.

In one preferred embodiment, the form of administration according to the invention is produced for administration twice daily, for administration once daily or for administration less frequently than once daily, preferably for administration not more than once daily. Administration is preferably systemic, in particular oral.

In one preferred embodiment, the form of administration according to the invention contains the compound according to the invention in such a small dose that it is not significantly effective in the treatment of acute pain. That dose is preferably in the range from 1.0 μg to 10 mg, based on the molecular weight of the free base.

Preferably, the dose is 0.001 mg±50%, 0.002 mg±50%, 0.003 mg±50%, 0.004 mg±50%, 0.005 mg±50%, 0.006 mg±50%, 0.007 mg±50%, 0.008 mg±50%, 0.009 mg±50%, 0.01 mg±50%, 0.02 mg±50%, 0.03 mg±50%, 0.04 mg±50%, 0.05 mg±50%, 0.06 mg±50%, 0.07 mg±50%, 0.08 mg±50%, 0.09 mg±50%, 0.1 mg±50%, 0.15 mg±50%, 0.2 mg±50%, 0.25 mg±50%, 0.3 mg±50%, 0.35 mg±50%, 0.4 mg±50%, 0.45 mg±50%, 0.5 mg±50%, 0.55 mg±50%, 0.6 mg±50%, 0.65 mg±50%, 0.7 mg±50%, 0.75 mg±50%, 0.8 mg±50%, 0.85 mg±50%, 0.9 mg±50%, 0.95 mg±50%, 1 mg±50%, 1.5 mg±50%, 2 mg±50%, 2.5 mg±50%, 3 mg±50%, 3.5 mg±50%, 4 mg±50%, 4.5 mg±50%, 5 mg±50%, 5.5 mg±50%, 6 mg±50%, 6.5 mg±50%, 7 mg±50%, 7.5 mg±50%, 8 mg±50%, 8.5 mg±50%, 9 mg±50%, 9.5 mg±50% or 10 mg±50%, based on the molecular weight of the free base.

More preferably, the dose is 0.001 mg±25%, 0.002 mg±25%, 0.003 mg±25%, 0.004 mg±25%, 0.005 mg±25%, 0.006 mg±25%, 0.007 mg±25%, 0.008 mg±25%, 0.009 mg±25%, 0.01 mg±25%, 0.02 mg±25%, 0.03 mg±25%, 0.04 mg±25%, 0.05 mg±25%, 0.06 mg±25%, 0.07 mg±25%, 0.08 mg±25%, 0.09 mg±25%, 0.1 mg±25%, 0.15 mg±25%, 0.2 mg±25%, 0.25 mg±25%, 0.3 mg±25%, 0.35 mg±25%, 0.4 mg±25%, 0.45 mg±25%, 0.5 mg±25%, 0.55 mg±25%, 0.6 mg±25%, 0.65 mg±25%, 0.7 mg±25%, 0.75 mg±25%, 0.8 mg±25%, 0.85 mg±25%, 0.9 mg±25%, 0.95 mg±25%, 1 mg±25%, 1.5 mg±25%, 2 mg±25%, 2.5 mg±25%, 3 mg±25%, 3.5 mg±25%, 4 mg±25%, 4.5 mg±25%, 5 mg±25%, 5.5 mg±25%, 6 mg±25%, 6.5 mg±25%, 7 mg±25%, 7.5 mg±25%, 8 mg±25%, 8.5 mg±25%, 9 mg±25%, 9.5 mg±25% or 10 mg±25%, based on the molecular weight of the free base.

Particularly preferably, the dose is 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg or 10 mg, based on the molecular weight of the free base.

In one preferred embodiment, the form of administration according to the invention contains the compound according to the invention in an amount of 10 μg±90%, more preferably 10 μg±75%, yet more preferably 10 μg±50%, most preferably 10 μg±25%, and in particular 10 μg±10%, based on the molecular weight of the free base.

In another preferred embodiment, the form of administration according to the invention contains the compound according to the invention in an amount of 100 μg±90%, more preferably 100 μg±75%, yet more preferably 100 μg±50%, most preferably 100 μg±25%, and in particular 100 μg±10%, based on the molecular weight of the free base.

In a further preferred embodiment, the form of administration according to the invention contains the compound according to the invention in an amount of 250 μg±90%, more preferably 250 μg±75%, yet more preferably 250 μg±50%, most preferably 250 μg±25%, and in particular 250 μg±10%, based on the molecular weight of the free base.

In a further preferred embodiment, the form of administration according to the invention contains the compound according to the invention in an amount of 500 μg±90%, more preferably 500 μg±75%, yet more preferably 500 μg±50%, most preferably 500 μg±25%, and in particular 500 μg±10%, based on the molecular weight of the free base.

In another preferred embodiment, the form of administration according to the invention contains the compound according to the invention in an amount of 750 μg±90%, more preferably 750 μg±75%, yet more preferably 750 μg±50%, most preferably 750 μg±25%, and in particular 750 μg±10%, based on the molecular weight of the free base.

In a further preferred embodiment, the form of administration according to the invention contains the compound according to the invention in an amount of 1000 μg±90%, more preferably 1000 μg±75%, yet more preferably 1000 μg±50%, most preferably 1000 μg±25%, and in particular 1000 μg±10%, based on the molecular weight of the free base.

The form of administration according to the invention can be administered, for example, as a liquid dosage form in the form of injection solutions, drops or juices, or as a semi-solid dosage form in the form of granules, tablets, pellets, patches, capsules, plasters/spray-on plasters or aerosols. The choice of auxiliary substances etc. and the amounts thereof to be used depend on whether the form of administration is to be administered orally, perorally, parenterally, intravenously, intraperitoneally, intradermally, intramuscularly, intranasally, buccally, rectally or locally, for example to the skin, the mucosa or into the eyes.

Forms of administration in the form of tablets, dragées, capsules, granules, drops, juices and syrups are suitable for oral administration, and solutions, suspensions, readily reconstitutable dry preparations and also sprays are suitable for parenteral, topical and inhalatory administration. Compounds according to the invention in a depot, in dissolved form or in a plaster, optionally with the addition of agents promoting penetration through the skin, are suitable percutaneous administration preparations.

Forms of administration which can be administered orally or percutaneously can release the compounds according to the invention in a delayed manner. The compounds according to the invention can also be administered in parenteral long-term depot forms, such as, for example, implants or implanted pumps. Other further active ingredients known to the person skilled in the art can in principle be added to the forms of administration according to the invention.

In one preferred embodiment, the compounds according to the invention are released from the form of administration immediately (immediate release, IR), that is to say preferably at least 80% of the active ingredient originally present is released under in vitro conditions, preferably according to Ph. Eur., after 20 minutes.

It has been found, surprisingly, that the compounds according to the invention are distinguished by an unusually long half-life (t_{1/2}) or pharmacodynamic duration of action, so that a comparatively infrequent administration is sufficient to achieve pharmacological effectiveness, and accordingly pain relief, which lasts a comparatively long time.

Forms of administration with prolonged release of the compounds according to the invention are not absolutely necessary therefor; a long-lasting action is achieved even in the case of immediate release (IR) because of the long half-life. The IR property of such forms of administration has the additional advantage that, with long-lasting effectiveness, rapid uptake of the active ingredient and accordingly a rapid onset of the pharmacological effectiveness after the first administration are nevertheless achieved. Accordingly, properties of IR forms of administration are combined with properties of PR forms of administration (PR, prolonged release).

In a preferred embodiment, the form of administration according to the invention is a form of administration with immediate release of the active ingredient (IR) which contains a compound according to the invention, preferably of the general formula (V) or (VI), in the form of the free base or a physiologically acceptable salt, preferably the hydrochloride, citrate or hemicitrate, and is produced preferably for oral administration not more than once daily, preferably exactly once daily. In this connection, “immediate release of the active ingredient” means that under in vitro conditions, preferably according to Ph. Eur., at least 80% of the active ingredient originally present has been released after 20 minutes.

The amount of the compounds according to the invention to be administered to the patient varies in dependence on the weight of the patient, on the type of administration, on the indication and on the severity of the disease. Usually, from 0.00005 to 50 mg/kg, preferably from 0.001 to 0.5 mg/kg, more preferably from 1 to 10 μg/kg, of at least one compound according to the invention is administered.

For all the above embodiments of the forms of administration according to the invention it is particularly preferred for the form of administration to contain a further active ingredient in addition to at least one compound according to the invention.

The ORL1 receptor and the μ-opioid receptor are associated in particular with the occurrence of pain. Accordingly, the compounds according to the invention can be used in the preparation of a medicament for the treatment of chronic pain, preferably of neuropathic pain, more preferably of mononeuropathic/neuralgic or polyneuropathic pain, more preferably of pain in the case of post-herpetic neuralgia or in the case of diabetic polyneuropathy.

The following examples serve to explain the invention but are not to be interpreted as being limiting.

In the following nomenclature of the stereochemistry of the example compounds, “(E)” refers to substitution on a double bond, for example on a cinnamic acid derivative, and “cis” and “trans” refer to substitution on the cyclohexyl ring.

### Synthesis of the Indole Structural Units (H)

### Structural Unit H-1:

### 2-(1H-indol-3-yl)ethanamine (H-1)

Available commercially at the time of the synthesis from Aldrich.

### Structural Unit H-2:

### 2-(5-Fluoro-1H-indol-3-yl)ethanamine (H-2)

Available commercially at the time of the synthesis from Fluorochem.

### Synthesis of the Ketone Structural Units (E)

Structural unit E-1:
Dimethyl-(8-phenyl-1,4-dioxaspiro[4.5]dec-8-yl)amine hydrochloride (D-1)

The aminonitrile B-1 (21 g, 0.1 mol), dissolved in THF (210 ml), was added in the course of 15 minutes, under argon and while cooling with ice, to a 1.82M phenylmagnesium chloride solution in THF (109 ml, 0.198 mol), and stirring was then carried out for 16 h at room temperature. For working up of the reaction mixture, saturated ammonium chloride solution (150 ml) was added, while cooling with ice, and extraction was carried out with diethyl ether (3×100 ml). The organic phase was extracted by shaking with water (100 ml) and saturated NaCl solution (100 ml) and concentrated. A yellow oil (25.2 g) remained. The crude product was dissolved in ethyl methyl ketone (280 ml), and ClSiMe₃ (18.8 ml, 0.15 mol) was added, while cooling with ice. After a reaction time of 6 h, the hydrochloride D-1 could be isolated in the form of a white solid in a yield of 35% (10.5 g).

4-Dimethylamino-4-phenylcyclohexanone (E-1)

The hydrochloride D-1 (10.5 g, 35.2 mmol) was dissolved in 7.5N hydrochloric acid (36 ml) and stirred for 96 h at room temperature. When hydrolysis was complete, the reaction mixture was extracted with diethyl ether (2×50 ml). While cooling with ice, the aqueous phase was rendered alkaline with 5N sodium hydroxide solution, extracted with dichloromethane (3×50 ml) and concentrated. The ketone 6 could thus be isolated in the form of a yellow solid having a melting point of 104-108° C. in a yield of 97% (7.4 g).

Structural unit E-2:

### Variant 1:

[8-(3-Fluorophenyl)-1,4-dioxaspiro[4.5]dec-8-yl]dimethylamine hydrochloride (D-2)

0.5M 3-fluorophenylmagnesium bromide solution in THF (3, 750 ml, 375 mmol) was added in the course of 15 minutes, under argon and while cooling with ice, to a solution of the aminonitrile B-1 (19.8 g, 94 mmol) in THF (100 ml), and stirring was then carried out for 16 h at room temperature. For working up of the reaction mixture, saturated ammonium chloride solution (150 ml) and water (60 ml) were added, while cooling with ice, and extraction was carried out with diethyl ether (3×100 ml). The organic phase was extracted by shaking with water (50 ml) and saturated NaCl solution (50 ml) and concentrated. There remained a brown oil (26.5 g), which in addition to the phenyl compound 4 also contained the ketal 2. The crude product was dissolved in ethyl methyl ketone (156 ml), and ClSiMe₃ (17.8 ml, 141 mmol) was added, while cooling with ice. After a reaction time of 6 h, the hydrochloride D-2 could be isolated in the form of a white solid having a melting point of 275-278° C. in a yield of 55% (16.3 g).

### Variant 2:

[8-(3-Fluoro-phenyl)-1,4-dioxa-spiro[4.5]dec-8-yl]-dimethyl-amine hydrochloride (D-2)

A solution of 1-bromo-3-fluorobenzene (5.00 g, 28.6 mmol) in abs. ether (15 ml) was added dropwise to a suspension of magnesium (694 mg, 28.6 mmol) in abs. ether (10 ml) in such a manner that the ether boiled. When the addition was complete, stirring was carried out for 10 min at RT, following which the magnesium was completely dissolved. The reaction solution was cooled in an ice bath, and the aminonitrile B-1 (3.00 g, 14.3 mmol) in abs. THF (30 ml) was added dropwise at 10° C. The batch was stirred overnight at room temperature; 20% NH₄Cl solution (20 ml) and water (30 ml) were added to the reaction mixture, while cooling with ice, and extraction was carried out with ether (3×50 ml). The organic phase was washed with water (50 ml) and then with saturated NaCl solution (50 ml), dried over Na₂SO₄ and concentrated in vacuo. The crude product was dissolved in ethyl methyl ketone (25 ml); ClSiMe₃ (3.2 ml, 25 mmol) was added, while cooling with ice, and stirring was carried out for 5 h at room temperature. The resulting precipitate was filtered off and dried in vacuo.

Yield of D-2: 2.8 g (62%)

¹H-NMR (DMSO-d₆): 1.91 (8H, m); 2.54 (6H, s); 3.91 (4H, d); 7.37 (1H, m); 7.61 (3H, m).

### Variant 1:

4-Dimethylamino-4-(3-fluoro-phenyl)-cyclohexanone (E-2)

The hydrochloride D-2 (7.2 g, 22.75 mmol) was dissolved in water (9.6 ml); concentrated hydrochloric acid (14 ml, 455 mmol) was added, and stirring was carried out for 4 d at room temperature. When hydrolysis was complete, the reaction mixture was extracted with diethyl ether (2×50 ml) and the aqueous phase was rendered alkaline with 5N sodium hydroxide solution, while cooling with ice, whereupon the product precipitated. The ketone E-2 could be isolated in the form of a yellow solid having a melting point of 83-88° C. in a yield of 50% (6.05 g).

### Variant 2:

4-Dimethylamino-4-(3-fluoro-phenyl)-cyclohexanone (E-2)

The hydrochloride D-2 (2.80 g, 8.86 mmol) was dissolved in water (3.7 ml); concentrated hydrochloric acid (5.5 ml) was added, and stirring was carried out for 4 d at RT. When hydrolysis was complete, the reaction mixture was extracted with ether (2×10 ml), the aqueous solution was rendered alkaline with 5N sodium hydroxide solution, while cooling with ice, the reaction mixture was extracted with dichloromethane (3×50 ml), and the organic phase was dried over sodium sulfate and concentrated in vacuo. The crude product was purified by flash chromatography with CHCl₃/MeOH (20:1).

Yield of E-2: 676 mg (32%), colourless solid

Melting point: 62-67° C.

¹H-NMR (DMSO-d₆): 2.02 (6H, s); 2.12 (5H, m); 2.45 (3H, m); 7.24 (3H, m); 7.43 (1H, m).

Structural unit E-3:
[8-(4-Fluorophenyl)-1,4-dioxaspiro[4.5]dec-8-yl]dimethylamine hydrochloride (D-3)

1M 4-fluorophenylmagnesium bromide solution in THF (3, 125 ml, 125 mmol) was added in the course of 15 min, under argon and while cooling with ice, to a solution of the aminonitrile B-1 (10.5 g, 50 mmol) in THF (150 ml), and stirring was then carried out for 16 h at room temperature. For working up of the reaction mixture, saturated ammonium chloride solution (37 ml) and water (50 ml) were added, while cooling with ice, and extraction was carried out with diethyl ether (3×100 ml). The organic phase was extracted by shaking with water (50 ml) and saturated NaCl solution (50 ml) and concentrated. There remained a brown oil (12.55 g) which contained, in addition to the phenyl compound C-3, also the ketal B-1. The crude product was dissolved in ethyl methyl ketone (75 ml), and ClSiMe₃ (9.5 ml, 75 mmol) was added, while cooling with ice. After a reaction time of 6 h, the hydrochloride D-3 could be isolated in the form of a white solid in a yield of 47% (7.48 g).

4-Dimethylamino-4-(4-fluorophenyl)cyclohexanone (E-3)

The hydrochloride D-3 (7.2 g, 22.75 mmol) was dissolved in water (9.6 ml); concentrated hydrochloric acid (14 ml, 455 mmol) was added, and stirring was carried out for 4 d at room temperature. When hydrolysis was complete, the reaction mixture was extracted with diethyl ether (2×50 ml) and the aqueous phase was rendered alkaline with 5N sodium hydroxide solution, while cooling with ice, extracted with dichloromethane (3×50 ml) and concentrated. The ketone E-3 could be isolated in the form of a yellow solid having a melting point of 128-133° C. in a yield of 76% (4.05 g).

Structural unit E-4:
Dimethyl-(8-thiophen-2-yl-1,4-dioxaspiro[4.5]dec-8-yl)amine hydrochloride (D-4)

2-lodothiophene (1, 22.9 g, 109 mmol) was dissolved, under argon, in THF (80 ml), and 2M isopropylmagnesium chloride (2, 35.7 ml, 72 mmol) in THF was added at 0° C. in the course of 30 min. After a reaction time of 1 h at 3-5° C., the aminonitrile B-1 (10 g, 47.6 mmol), dissolved in tetrahydrofuran (20 ml), was added, and stirring was carried out for 20 h at room temperature. Working up of the batch was carried out by addition of saturated NH₄Cl solution (85 ml) and extraction with diethyl ether (3×100 ml). The organic phase was extracted by shaking with water (50 ml) and saturated NaCl solution (50 ml) and concentrated. It was possible to obtain a dark-brown oil (21.3 g) which contained, in addition to the desired ketal, the aminonitrile B-1 and 2-iodothiophene. The crude product was dissolved in ethyl methyl ketone (140 ml), and ClSiMe₃ (9.1 ml, 71.4 mmol) was added. After a reaction time of 6 h, the hydrochloride D-4 was isolated in the form of a white crystalline compound in a yield of 60% (8.74 g).

4-Dimethylamino-4-thiophen-2-ylcyclohexanone (E-4)

The hydrochloride D-4 (8.68 g, 28.6 mmol) was dissolved in 7.5N hydrochloric acid (29 ml) and stirred for 48 h at room temperature. When hydrolysis was complete, the reaction mixture was extracted with diethyl ether (2×50 ml). The aqueous phase was rendered alkaline with 5N sodium hydroxide solution, while cooling with ice, extracted with dichloromethane (3×50 ml) and concentrated. The ketone E-4 was thus obtained in the form of a yellow solid having a melting point of 108-110° C. in a yield of 89 (5.66 g).

Structural unit E-5:
N,N-Dimethyl-8-(thiophen-3-yl)-1,4-dioxaspiro[4.5]decane-8-amine (D-5)

3-lodothiophene (1, 5 g, 23.8 mmol) was dissolved, under argon, in THF (18 ml), and 2M isopropylmagnesium chloride (2, 7.8 ml, 15.5 mmol) in THF was added in the course of 8 min at 0° C. After a reaction time of 1 h at 3-5° C., the aminonitrile B-1 (2, 16 g, 10.3 mmol), dissolved in tetrahydrofuran (20 ml), was added. Stirring was then carried out for 20 h at room temperature. Working up of the batch was carried out by addition of saturated NH₄Cl solution (20 ml) and extraction with diethyl ether (3×50 ml). The organic phase was extracted by shaking with water (20 ml) and saturated NaCl solution (20 ml) and concentrated. A light-brown oil (3.95 g) was obtained. The crude product was dissolved in ethyl methyl ketone (40 ml), and ClSiMe₃ (1.95 ml, 15.5 mmol) was added. After a reaction time of 3 h, the desired hydrochloride could be isolated in the form of a white crystalline compound in yield of 60% (1.86 g) with a melting point of 250-251° C.

### 4-(Dimethylamino)-4-(thiophen-3-yl)cyclohexanone (E-5)

The hydrochloride D-5 (1.8 g, 5.9 mmol) was dissolved in 7.5N hydrochloric acid (7 ml) and stirred for 48 h at room temperature. When hydrolysis was complete, the reaction mixture was extracted with diethyl ether (2×30 ml); while cooling with ice, the aqueous phase was rendered alkaline with 5N sodium hydroxide solution, extracted with dichloromethane (3×30 ml) and concentrated. The ketone E-5 could be isolated in the form of a yellow solid having a melting point of 147-150° C. in a yield of 98% (1.27 g).

### Synthesis of the Spiroamine Structural Units (AMN^{cis}/AMN^{trans})

### EXAMPLE AMN-1^{cis}

### 2′,3′,4′,9′-Tetrahydro-N,N-dimethyl-4-(phenyl)-spiro[cyclohexane-1,1′(1′H)-pyrido[3,4-b]indole]-4-amine (cis-diastereoisomer)

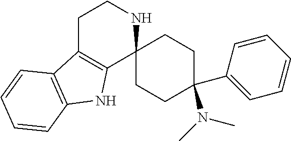 [SMILES: CN(C)[C@]1(c2ccccc2)CC[C@@]2(CC1)NCCc1c3ccccc3[nH]c12]

Note: According to this procedure, predominantly the cis product AMN-1^{cis} is obtained. The trans product AMN-1^{trans} is obtained only as a secondary product or in impure form.

The ketone E-1 (3.26 g, 15 mmol) and tryptamine H-1 (2.4 g, 15 mmol) were dissolved in dry MeOH (100 ml) with the exclusion of oxygen. Sodium sulfate (3 g) was added to that mixture. After a reaction time of 17 h, the solvent was distilled off in a rotary evaporator and the residue was taken up in 1,2-dichloroethane (100 ml). Trifluoroacetic acid (15 ml) was added to the reaction mixture and stirring was carried out for 1 h at room temperature. The progress of the reaction was monitored by TLC. For working up, H₂O (40 ml) was added to the batch and the pH was adjusted to 11 with NaOH (5 mol/l). A white solid precipitated and was filtered off with suction over a frit. The solid was washed with H₂O (3×5 ml) and dried. It was the cis product AMN-1^{cis}, which was obtained in the form of a white solid having a melting point of 214-218° C. in a yield of 4 g (74%). The mother liquor (aqueous phase) was extracted with 1,2-dichloroethane (3×25 ml). The organic phase was dried with Na₂SO₄ and concentrated. The solid brown residue was recrystallised from MeOH (10 ml) and yielded a mixture of cis-AMN-1^{cis} and trans-AMN-1^{trans} spiroamine (1:1). The mixture was obtained in the form of a white solid in a yield of 940 mg (17%).

¹H NMR (600 MHz, DMSO-d₆): 1.61 (m, 2H) 1.63 (m, 2H) 1.92 (s, 6H) 2.12 (m, 2H) 2.39 (m, 2H) 2.53 (t, J=5.36 Hz, 2H) 2.99 (t, J=5.35 Hz, 2H) 6.86 (m, 1H) 6.91 (m, 1H) 7.16 (d, J=7.52 Hz, 1H) 7.28 (d, J=7.52 Hz, 1H) 7.31 (m, 1H) 7.43 (m, 4H) 10.21 (s, 1H)

### EXAMPLE AMN-2^{cis}

### 2′,3′,4′,9′-Tetrahydro-N,N-dimethyl-4-(3-fluorophenyl)-spiro[cyclohexane-1,1′(1′H)-pyrido[3,4-b]indole]-4-amine (cis-diastereoisomer)

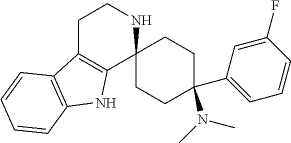 [SMILES: CN(C)[C@]1(c2cccc(F)c2)CC[C@@]2(CC1)NCCc1c3ccccc3[nH]c12]

The ketone E-2 (4.71 g, 20 mmol) and tryptamine H-1 (3.2 g, 20 mmol) were dissolved in dry MeOH (200 ml), under argon. After a reaction time of 24 h, MeOH was distilled off and the yellow, oily residue was suspended in 1,2-dichloroethane (200 ml). Trifluoroacetic acid (20 ml) was added to the reaction mixture and stirring was carried out for 2 h at room temperature. The progress of the reaction was monitored by TLC. For working up, the batch was diluted with H₂O (100 ml) and adjusted to pH 11 with NaOH (5 mol/l). After addition of ethyl acetate (50 ml), a white solid precipitated on stirring and was filtered off with suction over a frit. The solid was washed with H₂O (3×25 ml) and then dried. It was the cis-diastereoisomer AMN-2^{cis}, which was obtained in the form of a white solid having a melting point of 220-225° C. in a yield of 5.5 g (73%).

¹H NMR (600 MHz, DMSO-d₆): 1.61 (m, 2H) 1.62 (m, 2H) 1.93 (s, 6H) 2.11 (m, 2H) 2.38 (m, 2H) 2.53 (t, J=5.56 Hz, 2H) 2.99 (t, J=5.56 Hz, 2H) 6.87 (m, 1H) 6.92 (m, 1H) 7.14 (m, 1H) 7.17 (d, J=8.34 Hz, 1H) 7.20 (m, 1H) 7.25 (d, J=7.82 Hz, 1H) 7.28 (d, J=7.47 Hz, 1H) 7.47 (m, 1H) 10.26 (s, 1H)

### EXAMPLE AMN-2^{trans}

### 2′,3′,4′,9′-Tetrahydro-N,N-dimethyl-4-(3-fluorophenyl)-spiro[cyclohexane-1,1′(1′H)-pyrido[3,4-b]indole]-4-amine (trans-diastereoisomer)

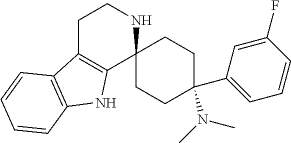 [SMILES: CN(C)[C@]1(c2cccc(F)c2)CC[C@]2(CC1)NCCc1c3ccccc3[nH]c12]

Tryptamine H-1 (2.03 g, 12.7 mmol) and the ketone (E-2, 3.0 g, 12.7 mmol) were dissolved in abs. methanol (130 ml) and stirred for 16 h at room temperature, under argon. The reaction mixture was then concentrated. The residue was dissolved in abs. 1,2-dichloroethane (130 ml); trifluoroacetic acid (12.7 ml) was added quickly, and stirring was carried out for 2 h at room temperature. While cooling with ice, water (120 ml) and 5N sodium hydroxide solution (40 ml) were added and stirring was carried out for 1 h. The colourless solid which formed thereby was separated off by filtration and washed with 1,2-dichloroethane (30 ml) and water (4×25 ml). The cis-spiroamine AMN-2^{cis} was obtained in a yield of 77% (3.7 g) with traces of the trans-spiroamine AMN-2^{trans}. The phases of the filtrate were separated. The organic phase was dried with sodium sulfate and concentrated, methanol (3 ml) was added, and stirring was carried out for 1 h at room temperature. A white solid precipitated and was separated off by filtration and washed with methanol (4×3 ml). The trans-spiroamine AMN-2^{trans} was obtained in a yield of 5% (250 mg) with traces of the cis-spiroamine AMN-2^{cis}. After purification by chromatography [silica gel 60 (20 g); methanol (200 ml)], the trans-spiroamine AMN-2^{trans} (170 mg) having a melting point of 296-299° C. was obtained.

¹H NMR (600 MHz, DMSO-d₆): 1.55 (m, 2H) 1.62 (m, 2H) 1.88 (s, 6H) 2.26 (m, 2H) 2.43 (m, 2H) 2.55 (t, J=5.49 Hz, 2H) 2.96 (t, J=5.25 Hz, 2H) 6.91 (m, 1H) 6.99 (m, 1H) 7.08 (m, 1H) 7.14 (m, 1H) 7.20 (d, J=7.64 Hz, 1H) 7.32 (m, 2H) 7.40 (m, 1H) 10.63 (s, 1H)

### EXAMPLE AMN-3^{cis}

### 6′-Fluoro-2′,3′,4′,9′-tetrahydro-N,N-dimethyl-4-(3-fluorophenyl)-spiro[cyclohexane-1,1′(1′H)-pyrido[3,4-b]indole]-4-amine (cis-diastereoisomer)

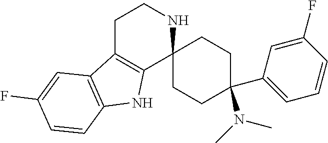 [SMILES: CN(C)[C@]1(c2cccc(F)c2)CC[C@@]2(CC1)NCCc1c3cc(F)ccc3[nH]c12]

The ketone E-2 (9.6 g, 41.2 mmol) and fluorotryptamine H-2 (7.3 g, 41.2 mmol) were dissolved in ethanol (200 ml) and heated for 12 hours at reflux. The ethanol was then distilled off and the crude product was suspended in 1,2-dichloroethane (100 ml). Trifluoroacetic acid (90 ml) was added to the reaction mixture and stirring was carried out for 12 h at room temperature. The progress of the reaction was monitored by TLC. For working up, the batch was rendered basic with 500 ml of 1N NaOH solution at 0° C. and then extracted 3× with 500 ml of ethyl acetate. The combined organic phases were dried over magnesium sulfate and concentrated under reduced pressure. After addition of methanol (100 ml), a white solid precipitated upon stirring and was filtered off with suction over a frit. The solid was washed with methanol (2×25 ml) and then dried. It was the cis-diastereoisomer AMN-3^{cis}, which was obtained in the form of a white solid in a yield of 3.6 g (22%).

¹H NMR (DMSO-d6, 400 MHz): δ 10.39 (s, 1H), 7.44-7.49 (m, 1H), 7.11-7.24 (m, 4H), 7.00-7.04 (m, 1H), 6.72-6.78 (m, 1H), 2.95-2.98 (t, 2H), 2.48-2.50 (m, 1H), 2.36-2.39 (d, 2H), 1.98-2.11 (m, 2H), 1.91 (s, 6H), 1.51-1.67 (m, 5H)

MS m/z (M+1): 396.4; Purity (HPLC): 95.03%

### EXAMPLE AMN-4^{cis}

### 6′-Fluoro-2′,3′,4′,9′-tetrahydro-N,N-dimethyl-4-(phenyl)-spiro[cyclohexane-1,1′(1′H)-pyrido[3,4-b]indole]-4-amine (cis-diastereoisomer)

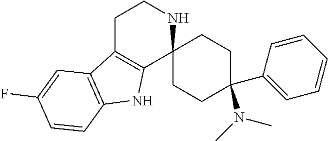 [SMILES: CN(C)[C@]1(c2ccccc2)CC[C@@]2(CC1)NCCc1c3cc(F)ccc3[nH]c12]

The ketone E-1 (8.4 g, 47 mmol) and fluorotryptamine H-2 (10.2 g, 47 mmol) were dissolved in ethanol (200 ml) and heated for 12 hours at reflux. The ethanol was then distilled off and the crude product was suspended in 1,2-dichloroethane (120 ml). Trifluoroacetic acid (100 ml) was added to the reaction mixture and stirring was carried out for 12 h at room temperature. The progress of the reaction was monitored by TLC. For working up, the batch was rendered basic with 1N NaOH solution at 0° C. and then extracted 3× with 500 ml of ethyl acetate. The combined organic phases were dried over magnesium sulfate and concentrated under reduced pressure. After addition of methanol (100 ml), a white solid precipitated upon stirring and was filtered off with suction over a frit. The solid was washed with methanol (2×25 ml) and then dried. It was the cis-diastereoisomer AMN-4^{cis}, which was obtained in the form of a white solid in a yield of 4 g (28%).

¹H NMR (DMSO-d₆, 400 MHz): δ 10.36 (s, 1H), 7.45-7.42 (t, 4H), 7.32-7.29 (m, 1H), 7.14-7.10 (m, 1H), 7.03-7.00 (m, 1H), 6.76-6.71 (m, 1H), 2.99-2.96 (t, 2H), 2.40-2.37 (d, 2H), 2.13-2.04 (m, 2H), 1.91 (s, 6H), 1.88 (s, 1H), 1.65-1.54 (m, 4H), 1.23 (s, 1H).

### EXAMPLE AMN-5^{cis}

### 2′,3′,4′,9′-Tetrahydro-N,N-dimethyl-4-(4-fluorophenyl)-spiro[cyclohexane-1,1′(1′H)-pyrido[3,4-b]indole]-4-amine (cis-diastereoisomer)

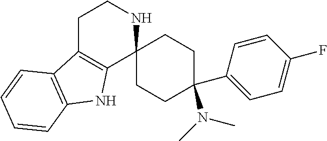 [SMILES: CN(C)[C@]1(c2ccc(F)cc2)CC[C@@]2(CC1)NCCc1c3ccccc3[nH]c12]

The ketone E-3 (2800 mg, 11.90 mmol) and tryptamine (H-1, 1910 mg, 11.90 mmol) were dissolved, under argon, in dry methanol (119 ml) and stirred for 18 h. The methanol was then distilled off in vacuo and the residue was suspended in 1,2-dichloroethane (119 ml). Trifluoroacetic acid (11.9 ml) was added to the reaction mixture and stirring was carried out for 2 h at room temperature. The reaction mixture was then diluted with 1,2-dichloroethane (119 ml) and adjusted to pH 11 with 1N sodium hydroxide solution, while cooling with ice. A pale precipitate formed. The mixture was stirred overnight at room temperature. The precipitate was filtered off with suction, washed with water and dried in vacuo. The cis-diastereoisomer AMN-5^{cis} (m.p. 249-250° C., in some cases 225-230° C.) could be isolated in a yield of 80% (3610 mg, 9.56 mmol). The phases were separated. The organic phase was dried with sodium sulfate, filtered and freed of volatile constituents in vacuo. The pale residue (trans-diastereoisomer AMN-5^{trans}) was taken up in methanol (5 ml) and stirred for 48 h. The precipitate was filtered off and dried in vacuo. The trans-diastereoisomer AMN-5^{trans} (268-271° C.) could be isolated in a yield of 6% (279 mg, 0.74 mmol).

¹³C{¹H}-NMR (101 MHz, DMSO-D₆) δ ppm: 22.8 (1C), 27.3 (2C), 32.6 (2C), 37.8 (2C), 38.6 (1C), 51.2 (1C), 60.5 (1C), 106.7 (1C), 110.8 (1C), 114.2 (2C, d, J=21 Hz), 117.2 (1C), 117.9 (1C), 120.0 (1C), 126.9 (1C), 129.7 (2C, d, J=8 Hz), 132.8 (1C, d, J=3 Hz), 135.4 (1C), 141.4 (1C), 160.7 (1C, d, J=242 Hz)

### Synthesis of the Cis-Spiroamide Examples (AMD^{cis})

### EXAMPLE AMD-1^{cis}

### (E)-2′,3′,4′,9′-Tetrahydro-N,N-dimethyl-4-phenyl-2′-(2-phenylvinyl)carbonyl-spiro[cyclohexane-1,1′(1′H)-pyrido[3,4-b]indole]-4-amine methanesulfonate (1:1) (cis-diastereoisomer)

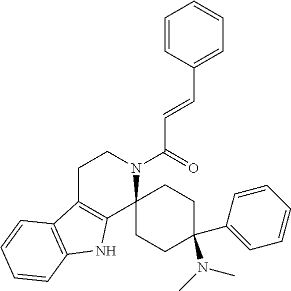 [SMILES: CN(C)[C@]1(c2ccccc2)CC[C@]2(CC1)c1[nH]c3ccccc3c1CCN2C(=O)/C=C/c1ccccc1]

AMN-1^{cis} was dissolved in THF (8 ml). Cinnamic acid chloride (254 mg, 1.53 mmol) and diisopropylethylamine (216 mg, 1.67 mmol) were then added, and stirring was carried out for 2 d at RT. When the reaction was complete, the solid was filtered off and saturated Na₂CO₃ solution was added to the filtrate. The aqueous phase was extracted three times with 10 ml of ethyl acetate each time. The organic phase was then dried over MgSO₄ and concentrated in a rotary evaporator. The crude product was purified by column chromatography [silica gel 60; DCM/methanol (19:1, 570 ml)]. The product was obtained in a yield of 174 mg (26%). In order to prepare the methanesulfonate, the spiroamide just obtained (174 mg, 0.355 mmol) was suspended in DCM (6 ml), and methanesulfonic acid (23.7 μl, 0.355 mmol) was added at RT. Acetone (0.8 ml) was then added, and sufficient diethyl ether was added to disperse the cloudiness that occurred by shaking. Stirring was carried out for a further 30 min and the resulting solid was then filtered off with suction, with the exclusion of air, washed with diethyl ether and dried for 3 h at 50° C. under an oil pump vacuum. The product AMD-1^{cis} was obtained in a yield of 159 mg (76%).

¹H NMR (600 MHz, DMSO-d₆) 1.65 (t, J=13.22 Hz, 2H) 2.20 (t, J=12.84 Hz, 2H) 2.51 (d, J=4.53 Hz, 9H) 2.87-3.16 (m, 4H) 4.13 (br. s., 2H) 6.92 (t, J=7.55 Hz, 1H) 6.99 (t, J=7.55 Hz, 1H) 7.20 (d, J=8.31 Hz, 1H) 7.31 (d, J=7.55 Hz, 1H) 7.36-7.51 (m, 5H) 7.56-7.69 (m, 3H) 7.74 (d, J=7.55 Hz, 2H) 7.82 (d, J=7.55 Hz, 2H) 9.62 (br, s, 1H)

### EXAMPLE AMD-2^{cis}

### 2′,3′,4′,9′-Tetrahydro-N,N-dimethyl-4-(3-fluoro-phenyl)-2′-(4-chlorobenzyl)-carbonyl-spiro[cyclohexane-1,1′(1′H)-pyrido[3,4-b]indole]-4-amine (cis-diastereoisomer)

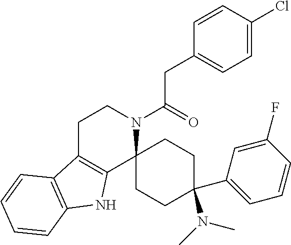 [SMILES: CN(C)[C@]1(c2cccc(F)c2)CC[C@]2(CC1)c1[nH]c3ccccc3c1CCN2C(=O)Cc1ccc(Cl)cc1]

The spiroamine (AMN-2^{cis}; 396 mg, 1.05 mmol) was suspended in DCM (15 ml) in a vessel suitable for microwaves, and 2-(4-chlorophenyl)acetyl chloride (397 mg, 2.1 mmol) and diisopropylethylamine (269 mg, 2.1 mmol) were added. The reaction mixture was irradiated for 10 min at 120° C. in a microwave (Initiator Eight, Biotage). When the reaction was complete (TLC monitoring), the reaction mixture was first filtered, diethyl ether (15 ml) was added, and filtering was carried out again. Saturated Na₂CO₃ solution (8 ml) was added. After separation of the phases, the aqueous phase was washed again with DCM. The combined organic phases were dried over MgSO₄ and concentrated in a rotary evaporator. The crude product was purified by column chromatography [silica gel 60; DCM/methanol (19:1)]. The product AMD-2^{cis} was obtained in a yield of 91 mg (16%).

¹H NMR (600 MHz, DMSO-d₆) δ ppm 1.55 (t, J=13.60 Hz, 2H) 1.79 (t, J=12.84 Hz, 2H) 1.92 (br. s., 6H) 2.60-2.70 (m, 2H) 2.73-2.87 (m, 2H) 3.17 (d, J=5.29 Hz, 2H) 3.89-4.01 (m, 4H) 6.90 (t, J=7.55 Hz, 1H) 6.97 (t, J=7.55 Hz, 1H) 7.08-7.16 (m, 1H) 7.18 (d, J=8.31 Hz, 1H) 7.21-7.30 (m, 3H) 7.34 (q, J=8.31 Hz, 4H) 7.46 (q, J=7.30 Hz, 1H) 10.53 (s, 1H)

### EXAMPLE AMD-3^{cis}

### 2′,3′,4′,9′-Tetrahydro-N,N-dimethyl-4-(3-fluorophenyl)-2′-(benzothiophen-2-yl)carbonyl-spiro[cyclohexane-1,1′(1′H)-pyrido[3,4-b]indole]-4-amine (cis-diastereoisomer)

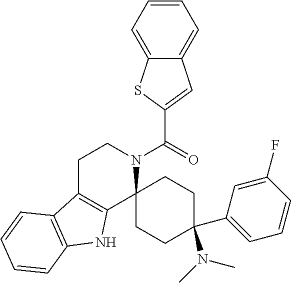 [SMILES: CN(C)[C@]1(c2cccc(F)c2)CC[C@]2(CC1)c1[nH]c3ccccc3c1CCN2C(=O)c1cc2ccccc2s1]

The spiroamine (AMN-2^{cis}; 264 mg, 0.7 mmol) was suspended in DCM (7 ml) in a vessel suitable for microwaves, and benzo[b]thiophene-2-carbonyl chloride (239 mg, 1.21 mmol) and diisopropylethylamine (180 mg, 1.4 mmol) were added. The reaction mixture was irradiated for 10 min at 100° C. in a microwave (Initiator Eight, Biotage). When the reaction was complete (TLC monitoring), the reaction mixture was diluted with DCM (15 ml) and filtered. Saturated Na₂CO₃ solution (8 ml) was added to the mother liquor. After separation of the phases, the aqueous phase was washed twice more with DCM. The combined organic phases were dried over MgSO₄ and concentrated in a rotary evaporator. The crude product was purified by column chromatography [silica gel 60; DCM/methanol (19:1)]. The product AMD-3^{cis} was obtained in a yield of 125 mg (33%).

¹H NMR (600 MHz, DMSO-d₆) δ ppm 1.63-1.79 (m, 2H) 1.83-1.93 (m, 2H) 1.95 (s, 6H) 2.60 (d, J=13.60 Hz, 2H) 2.65 (t, J=5.67 Hz, 2H) 2.78-2.94 (m, 2H) 4.08-4.22 (m, 2H) 6.92 (t, J=7.55 Hz, 1H) 6.99 (t, J=7.55 Hz, 1H) 7.16 (t, J=8.31 Hz, 1H) 7.23 (d, J=8.31 Hz, 1H) 7.26-7.36 (m, 3H) 7.44-7.54 (m, 3H) 7.95 (s, 1H) 8.03 (d, J=7.55 Hz, 1H) 8.07 (d, J=8.31 Hz, 1H) 10.66 (s, 1H)

### EXAMPLE AMD-4^{cis}

### 2′,3′,4′,9′-Tetrahydro-N,N-dimethyl-4-(3-fluoro-phenyl)-2′-(4-fluorobenzyl)-carbonyl-spiro[cyclohexane-1,1′(1′H)-pyrido[3,4-b]indole]-4-amine methanesulfonate (cis-diastereoisomer)

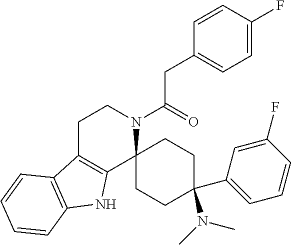 [SMILES: CN(C)[C@]1(c2cccc(F)c2)CC[C@]2(CC1)c1[nH]c3ccccc3c1CCN2C(=O)Cc1ccc(F)cc1]

The spiroamine (AMN-2^{cis}; 600 mg, 1.59 mmol) was suspended in DCM (15 ml) in a vessel suitable for microwaves, and 2-(4-fluorophenyl)acetyl chloride (548 mg, 3.18 mmol) and diisopropylethylamine (408 mg, 3.18 mmol) were added. The reaction mixture was irradiated for 10 min at 130° C. in a microwave (Initiator Eight, Biotage). When the reaction was complete (TLC monitoring), the reaction mixture was first filtered, the mother liquor was diluted with DCM (45 ml), and saturated Na₂CO₃ solution (25 ml) was added. After separation of the phases, the organic phase was washed again with saturated Na₂CO₃ solution. The organic phase was dried over MgSO₄ and concentrated in a rotary evaporator. The crude product was purified by column chromatography [silica gel 60; DCM/methanol (4:1)]. The product was obtained in a yield of 150 mg (18%). In order to prepare the methanesulfonate, the spiroamide (150 mg, 0.29 mmol) was dissolved in DCM (1 ml), and methanesulfonic acid (18.9 μl, 0.29 mmol) was added at RT. The mixture was diluted with diethyl ether so that a stirrable mixture formed. The solid was filtered off with suction, with the exclusion of air, washed with diethyl ether and dried at 50° C. under an oil pump vacuum. The product AMD-4^{cis} was obtained in a yield of 148 mg (83%).

¹H NMR (600 MHz, DMSO-d₆) δ ppm 1.58 (t, J=12.84 Hz, 2H) 2.16 (t, J=12.09 Hz, 2H) 2.31 (s, 3H) 2.53-2.58 (m, 6H) 2.58-2.68 (m, 2H) 2.83-3.03 (m, 4H) 3.98 (s, 2H) 3.99-4.06 (m, 2H) 6.92 (t, J=7.18 Hz, 1H) 6.99 (t, J=7.18 Hz, 1H) 7.14 (t, J=8.31 Hz, 2H) 7.18 (d, J=8.31 Hz, 1H) 7.29 (d, J=7.55 Hz, 1H) 7.36 (t, J=6.42 Hz, 2H) 7.45 (t, J=7.93 Hz, 1H) 7.58-7.75 (m, 3H) 9.65 (br. s., 1H)

### EXAMPLE AMD-5^{cis}

### (E)-2′,3′,4′,9′-Tetrahydro-N,N-dimethyl-4-(3-fluorophenyl)-2′-(2-phenylvinyl)carbonyl-spiro[cyclohexane-1,1′(1′H)-pyrido[3,4-b]indole]-4-amine (cis-diastereoisomer)

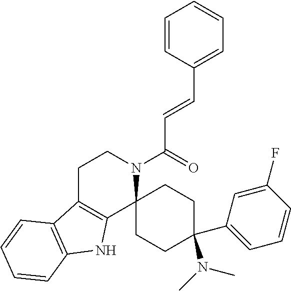 [SMILES: CN(C)[C@]1(c2cccc(F)c2)CC[C@]2(CC1)c1[nH]c3ccccc3c1CCN2C(=O)/C=C/c1ccccc1]

The spiroamine (AMN-2^{cis}; 378 mg, 1.0 mmol) was dissolved in dry aprotic solvent (6 ml); cinnamoyl chloride (183 mg, 1.1 mmol) and diisopropylethylamine (155 mg, 1.2 mmol) were added, and stirring was carried out overnight at RT. When the reaction was complete (TLC monitoring), the solvent was removed, the residue was subjected to aqueous working-up, and extraction was carried out with halogenated solvent. The combined organic phases were dried over Na₂SO₄ and concentrated. The crude product was purified by column chromatography. During the concentration, a solid precipitated and was filtered off and then dried. The product was obtained in a yield of 220 mg (43%).

¹H NMR (600 MHz, DMSO-d₆) δ ppm 1.63 (t, J=13.60 Hz, 2H) 1.84 (t, J=13.22 Hz, 2H) 1.91 (s, 6H) 2.54-2.63 (m, 2H) 2.65 (t, J=5.67 Hz, 2H) 2.82-3.02 (m, 2H) 3.17 (d, J=5.29 Hz, 2H) 4.00-4.22 (m, 2H) 6.90 (t, J=7.18 Hz, 1H) 6.97 (t, J=7.55 Hz, 1H) 7.11-7.18 (m, 1H) 7.20 (d, J=8.31 Hz, 1H) 7.23-7.33 (m, 3H) 7.35-7.54 (m, 5H) 7.72 (d, J=6.80 Hz, 2H) 10.59 (s, 1H)

### EXAMPLE AMD-6^{cis}

### (E)-2′,3′,4′,9′-Tetrahydro-N,N-dimethyl-4-(3-fluorophenyl)-2′-(2-phenylvinyl)carbonyl-spiro[cyclohexane-1,1′(1′H)-pyrido[3,4-b]indole]-4-amine citrate (cis-diastereoisomer)

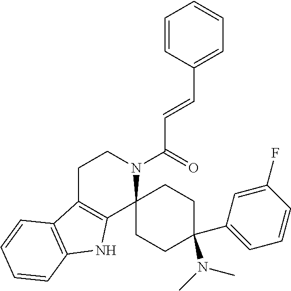 [SMILES: CN(C)[C@]1(c2cccc(F)c2)CC[C@]2(CC1)c1[nH]c3ccccc3c1CCN2C(=O)/C=C/c1ccccc1]

In order to prepare the salt, the amide AMD-5^{cis} (220 mg, 0.43 mmol) was dissolved in dry aprotic solvent (1.5 ml), and citric acid (83 mg, 0.43 mmol) dissolved in as little protic solvent as possible was added. In order to precipitate the product, non-polar solvent was added dropwise. The solid was then filtered off with suction, with the exclusion of air, and dried at 50° C. under an oil pump vacuum. The product AMD-6^{cis} was obtained in a yield of 100 mg (33%).

### EXAMPLE AMD-7^{cis}

### 2′,3′,4′,9′-Tetrahydro-N,N-dimethyl-4-(3-fluorophenyl)-2′-(3,4-dimethoxybenzyl)carbonyl-spiro[cyclohexane-1,1′(1′H)-pyrido[3,4-b]indole]-4-amine (cis-diastereoisomer)

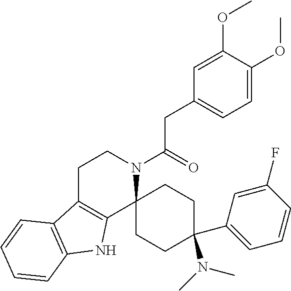 [SMILES: COc1ccc(CC(=O)N2CCc3c4ccccc4[nH]c3[C@]23CC[C@](c2cccc(F)c2)(N(C)C)CC3)cc1OC]

The spiroamine (AMN-2^{cis}; 200 mg, 0.54 mmol) was suspended in halogenated solvent (5 ml) in a vessel suitable for microwaves, and 2-(3,4-dimethoxyphenyl)acetyl chloride (230 mg, 1.1 mmol) and diisopropylethylamine (138 mg, 1.1 mmol) were added. The reaction mixture was irradiated for 10 min at 120° C. in a microwave (Initiator Eight, Biotage). When the reaction was complete (TLC monitoring), the reaction mixture was first filtered and then NaOH solution (5 N, 10 ml) was added to the mother liquor. After separation of the phases, the aqueous phase was extracted three times with a polar, aprotic solvent (in each case 5 ml). The combined organic phases were dried over MgSO₄ and concentrated. The crude product was purified by column chromatography. The product AMD-7^{cis} was obtained in a yield of 140 mg (47%).

¹H NMR (600 MHz, DMSO-d₆) δ ppm 1.54 (t, J=12.46 Hz, 2H) 1.79 (t, J=13.22 Hz, 2H) 1.85-1.96 (m, 6H) 2.52-2.60 (m, 2H) 2.62-2.72 (m, 2H) 2.73-2.89 (m, 2H) 3.74 (s, 3H) 3.77 (s, 3H) 3.82 (br. s., 2H) 3.90 (br. s., 2H) 6.83-6.93 (m, 4H) 6.97 (t, J=7.55 Hz, 1H) 7.13 (t, J=7.18 Hz, 1H) 7.19 (d, J=8.31 Hz, 1H) 7.20-7.32 (m, 3H) 7.41-7.54 (m, 1H) 10.53 (s, 1H)

### EXAMPLE AMD-8^{cis}

(E)-2′,3′,4′,9′-Tetrahydro-N,N-dimethyl-6′-fluoro-4-(3-fluorophenyl)-2′-(2-phenylvinyl)carbonyl-spiro[cyclohexane-1,1′(1′H)-pyrido[3,4-b]indole]-4-amine (cis-diastereoisomer)

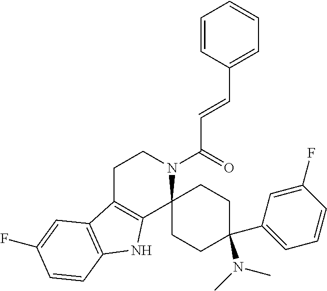 [SMILES: CN(C)[C@]1(c2cccc(F)c2)CC[C@]2(CC1)c1[nH]c3ccc(F)cc3c1CCN2C(=O)/C=C/c1ccccc1]

A suspension of the spiroamine AMN-3^{cis} (0.197 g; 0.5 mmol; 1 eq.) in 15 ml of abs. DCM was placed in a microwave vessel. Ethyl-diisopropylamine (0.129 g; 1 mmol; 2 eq.) and cinnamic acid chloride (0.166 g; 1 mmol; 2 eq.) were added in succession to that suspension. The microwave vessel was closed and heated for 10 min at 120° C. in a microwave (Initiator Eight, Biotage). For working up, 4 ml of water and 4 ml of 1N sodium hydroxide solution were added to the reaction mixture. The mixture was stirred for 2 h at RT. Then the phases were separated and the aqueous phase was extracted 3× with DCM. The combined organic phases were washed with water and dried over sodium sulfate. After the solvent had been removed at reduced pressure, the residue was purified by column chromatography (silica gel; ethyl acetate/cyclohexane 1:2→1:0). 0.087 g of product AMD-8^{cis} (33%) was obtained.

HPLC/MS analysis: Rₜ=4.2 min; Purity (UV 200-400 nm) 97%; m/z=526.1

### EXAMPLE AMD-9^{cis}

### 2′,3′,4′,9′-Tetrahydro-N,N-dimethyl-6′-fluoro-4-(3-fluorophenyl)-2′-(benzyl)carbonyl-spiro[cyclohexane-1,1′(1′H)-pyrido[3,4-b]indole]-4-amine (cis-diastereoisomer)

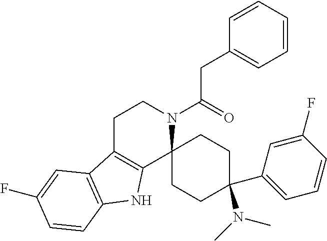 [SMILES: CN(C)[C@]1(c2cccc(F)c2)CC[C@]2(CC1)c1[nH]c3ccc(F)cc3c1CCN2C(=O)Cc1ccccc1]

A suspension of the spiroamine AMN-3^{cis} (0.25 g; 0.63 mmol; 1 eq.) in 19 ml of abs. DCM was placed in a microwave vessel. Ethyl-diisopropylamine (0.163 g; 1.26 mmol; 2 eq.) and 2-phenylacetyl chloride (0.195 g; 1.26 mmol; 2 eq.) were added in succession to that suspension. The microwave vessel was closed and heated for 10 min at 120° C. in a microwave (Initiator Eight, Biotage). For working up, 5 ml of water and 5 ml of 1N sodium hydroxide solution were added to the reaction mixture. The mixture was stirred for 2 h at RT. Then the phases were separated and the aqueous phase was extracted 3× with DCM. The combined organic phases were washed with water and dried over sodium sulfate. After the solvent had been removed at reduced pressure, the residue was purified by column chromatography (silica gel; ethyl acetate→ethyl acetate/methanol 9:1). 0.145 g of product AMD-9^{cis} (45%) was obtained.

HPLC/MS analysis: Rₜ=3.9 min; Purity (UV 200-400 nm) 98%; m/z=514.1

### EXAMPLE AMD-10^{cis}

### (E)-2′,3′,4′,9′-Tetrahydro-N,N-dimethyl-6′-fluoro-4-phenyl-2′-(2-phenylvinyl)carbonyl-spiro[cyclohexane-1,1′(1′H)-pyrido[3,4-b]indole]-4-amine (cis-diastereoisomer)

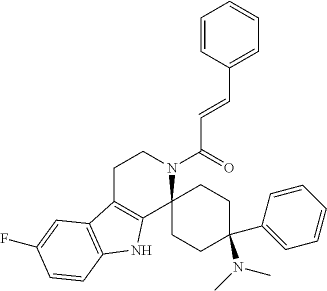 [SMILES: CN(C)[C@]1(c2ccccc2)CC[C@]2(CC1)c1[nH]c3ccc(F)cc3c1CCN2C(=O)/C=C/c1ccccc1]

A solution of the spiroamine AMN-4^{cis} (0.15 g; 0.397 mmol; 1 eq.) in 9 ml of abs. THF was added under nitrogen at RT to a solution of cinnamic acid chloride (0.198 g; 1.192 mmol; 3 eq.) in 4.5 ml of abs. THF. After stirring for 1 h at RT, first 3 ml of water and, while cooling with ice, 3 ml of 1N sodium hydroxide solution were added to the cloudy reaction solution. Stirring was carried out for 1.5 h. After the solvent had been removed at reduced pressure, the resulting solid was filtered off and washed with water. The crude product was purified by column chromatography (silica gel; ethyl acetate). 0.043 g of product AMD-10^{cis} (21%) was obtained.

HPLC/MS analysis: Rₜ=4.2 min; Purity (UV 200-400 nm) 98%; m/z=508.2

### EXAMPLE AMD-11^{cis}

### 2′,3′,4′,9′-Tetrahydro-N,N-dimethyl-4-(3-fluorophenyl)-2′-benzylcarbonyl-spiro[cyclohexane-1,1′(1′H)-pyrido[3,4-b]indole]-4-amine (cis-diastereoisomer)

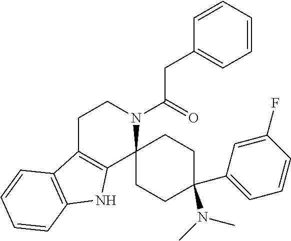 [SMILES: CN(C)[C@]1(c2cccc(F)c2)CC[C@]2(CC1)c1[nH]c3ccccc3c1CCN2C(=O)Cc1ccccc1]

The cis-spiroamine AMN-2^{cis} (1.29 g, 3.4 mmol) was dissolved, with the exclusion of oxygen, in absolute tetrahydrofuran (20 ml) and absolute dichloromethane (120 ml); Hünig base (1.167 ml, 6.8 mmol) was added, and 2-phenylacetyl chloride (900 μl, 6.8 mmol) was added at room temperature. After a reaction time of 30 min, 5N sodium hydroxide solution (100 ml) was added to the mixture, and stirring was carried out for 2 h. The aqueous phase was separated off and extracted with dichloromethane (3×10 ml). The combined organic phases were dried over Na₂SO₄ and then concentrated. A crude product was isolated and was separated by chromatography [silica gel 60 (100 g); EtOAc (1000 ml)]. The cis-amide AMD-11^{cis} was obtained in the form of a colourless solid in a yield of 820 mg (49%) with a melting point of 95-100° C.

¹³C-NMR (101 MHz, DMSO-D₆) δ ppm: 22.1, 29.1, 33.0, 38.0, 40.8, 43.1, 60.0, 60.3, 105.5, 111.1, 113.7, 113.2, 114.5, 114.7, 117.3, 118.4, 120.5, 123.8, 126.2, 126.5, 128.2, 129.0, 129.2, 129.3, 135.3, 136.5, 139.5, 140.6, 161.1, 163.5, 173.4

### EXAMPLE AMD-12^{cis}

### (E)-2′,3′,4′,9′-Tetrahydro-N,N-dimethyl-4-(4-fluorophenyl)-2′-(2-phenylvinyl)carbonyl-spiro[cyclohexane-1,1′(1′H)-pyrido[3,4-b]indole]-4-amine (cis-diastereoisomer)

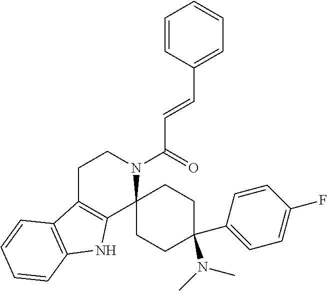 [SMILES: CN(C)[C@]1(c2ccc(F)cc2)CC[C@]2(CC1)c1[nH]c3ccccc3c1CCN2C(=O)/C=C/c1ccccc1]

Hünig base (0.45 ml, 342 mg, 2.64 mmol) and cinnamic acid chloride (440 mg, 2.64 mmol), dissolved in absolute dichloromethane (12 ml), were added dropwise in succession in the course of 10 min, under argon, to a suspension of the cis-spiroamine AMN-5^{cis} (500 mg, 1.32 mmol). The reaction mixture was stirred for 1 h at room temperature, and then water (30 ml) and 1N sodium hydroxide solution (5 ml) were added and stirring was carried out for 1.5 h. The dichloromethane was then removed in vacuo. A pale solid precipitated and was separated off by filtration and then washed with water (3×30 ml). The crude product so obtained was purified by chromatography [silica gel 60 (70 g), ethyl acetate/cyclohexane 1:1 (500 ml), ethyl acetate (1000 ml), ethyl acetate/methanol 10:1 (330 ml), ethyl acetate/methanol 4:1 (800 ml), methanol (300 ml)]. For application of the crude product to the column, it was necessary to dissolve the reaction product in ethyl acetate/cyclohexane 1:1 with a small amount of tetrahydrofuran. The cis-amide AMD-12^{cis} (m.p. 145-155° C.) was obtained in the form of a colourless solid in a yield of 31% (204 mg, 0.40 mmol).

¹³C{¹H}-NMR (101 MHz, DMSO-D₆) δ ppm: 22.5 (1C), 29.3 (2C), 32.6 (2C), 37.8 (2C), 41.3 (1C), 59.5 (1C), 60.3 (1C, br), 105.4 (1C), 111.1 (1C), 114.3 (2C, d, J=20 Hz), 117.3 (1C), 118.4 (1C), 120.5 (1C), 123.1 (1C), 126.6 (1C), 127.9 (2C), 128.7 (2C), 129.3 (2C), 129.8 (2C, d, J=8 Hz), 132.4 (1C, br), 135.1 (1C), 135.4 (1C), 139.4 (1C), 140.4 (1C), 160.9 (1C, d, J=243 Hz), 170.3 (1C)

### Synthesis of the Trans-Spiroamide Comparison Examples (AMD^{trans})

### EXAMPLE AMD-3^{trans}

### 2′,3′,4′,9′-Tetrahydro-N,N-dimethyl-4-(3-fluorophenyl)-2′-(benzothiophen-2-yl)carbonyl-spiro[cyclohexane-1,1′(1′H)-pyrido[3,4-b]indole]-4-amine citrate (1:1) (trans-diastereoisomer)

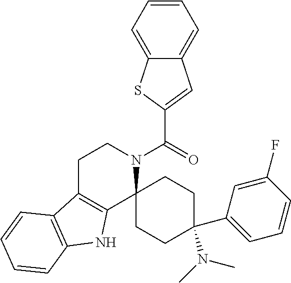 [SMILES: CN(C)[C@]1(c2cccc(F)c2)CC[C@@]2(CC1)c1[nH]c3ccccc3c1CCN2C(=O)c1cc2ccccc2s1]

### 2′,3′,4′,9′-Tetrahydro-N,N-dimethyl-4-(3-fluorophenyl)-2′-(benzothiophen-2-yl)carbonyl-spiro[cyclohexane-1,1′(1′H)-pyrido[3,4-b]indole]-4-amine (trans-diastereoisomer)

Benzo[b]thiophene-2-carboxylic acid chloride (728 mg, 3.96 mmol) was dissolved, under argon, in abs. tetrahydrofuran (30 ml), and the trans-spiroamine AMN-2^{trans} (500 mg, 1.32 mmol), dissolved in abs. tetrahydrofuran (60 ml), was added in the course of 75 min at room temperature. A slight precipitate formed. After a reaction time of 2 h, the reaction mixture was diluted with water (15 ml); 1N sodium hydroxide solution (15 ml) was added, while cooling with ice, and stirring was carried out for 2.5 h. Tetrahydrofuran was removed in vacuo. A solid formed and was separated out by filtration and washed with water (3×20 ml). The crude product (587 mg) was separated by chromatography [silica gel 60 (80 g); ethyl acetate/cyclohexane 1:1 (1 l), ethyl acetate/methanol 4:1 (500 ml)]. The trans-amide was thus obtained in the form of a colourless solid in a yield of 12% (82 mg) with a melting point of 219-221° C.

¹³C-NMR (101 MHz, CDCl₃) δ ppm: 22.4, 30.0, 30.9, 38.2, 46.4, 58.3, 59.5, 106.2, 111.0, 113.5, 113.7, 114.4, 114.7, 118.0, 119.1, 121.4, 122.5, 123.1, 124.7, 125.5, 125.8, 126.4, 128.7, 136.0, 138.7, 140.1, 140.4, 141.1, 142.1, 161.2, 163.7, 167.1

### 2′,3′,4′,9′-Tetrahydro-N,N-dimethyl-4-(3-fluorophenyl)-2′-(benzothiophen-2-yl)carbonyl-spiro[cyclohexane-1,1′(1′H)-pyrido[3,4-b]indole]-4-amine citrate (1:1) (trans-diastereoisomer; AMD-3^{trans})

The trans-amide just prepared (82 mg, 0.152 mmol) was suspended at 80° C. in ethanol (8 ml), and an ethanolic solution (3 ml) of citric acid (32 mg, 0.167 mmol) was added. On cooling to room temperature, a solid precipitated from the clear solution. After 1.5 h, the mixture was concentrated to 2 ml, diethyl ether (20 ml) was added, and stirring was carried out for 20 min. A colourless solid was separated off by filtration and washed with diethyl ether (2×3 ml) (64 mg). After 3 days, further solid had precipitated from the filtrate at room temperature and was filtered off with suction and washed with diethyl ether (2×2 ml) (35 mg). The two fractions were combined. The trans-citrate AMD-3^{trans} was thus obtained in a yield of 81% (89 mg) with a melting point of 175-185° C.

### EXAMPLE AMD-6^{trans}

### (E)-2′,3′,4′,9′-Tetrahydro-N,N-dimethyl-4-(3-fluorophenyl)-2′-(2-phenylvinyl)carbonyl-spiro[cyclohexane-1,1′(1′H)-pyrido[3,4-b]indole]-4-amine citrate (1:1) (trans-diastereoisomer)

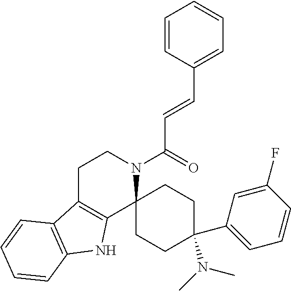 [SMILES: CN(C)[C@]1(c2cccc(F)c2)CC[C@@]2(CC1)c1[nH]c3ccccc3c1CCN2C(=O)/C=C/c1ccccc1]

### 2′,3′,4′,9′-Tetrahydro-N,N-dimethyl-4-(3-fluorophenyl)-2′-(2-phenylvinyl)carbonyl-spiro[cyclohexane-1,1′(1′H)-pyrido[3,4-b]indole]-4-amine (trans-diastereoisomer)

Cinnamic acid chloride (1.32 g, 7.92 mmol) was dissolved under argon in abs. tetrahydrofuran (30 ml), and impure spiroamine AMN-2^{cis} (1.0 g, 2.64 mmol, contains almost 10% trans-diastereoisomer AMN-2^{trans}), dissolved in abs. tetrahydrofuran (60 ml), was added in the course of 40 min, at room temperature. After a reaction time of 1 h, water (20 ml) and, while cooling with ice, 1N sodium hydroxide solution (20 ml) were added to the cloudy reaction solution, and stirring was carried out for 1.5 h. Tetrahydrofuran was removed in vacuo. A solid precipitated and was separated off by filtration and washed with water (3×25 ml). The crude product (1.16 g) was separated by chromatography [silica gel 60 (200 g); ethyl acetate/cyclohexane 1:1 (1.3 l), ethyl acetate (1.6 l)]. The cis-amide was obtained in the form of a colourless solid in a yield of 40% (540 mg) with a melting point of 155-158° C. The trans-amide was isolated in a yield of 7% (93 mg) with a melting point of 151-155° C.

### 2′,3′,4′,9′-Tetrahydro-N,N-dimethyl-4-(3-fluorophenyl)-2′-(2-phenylvinyl)carbonyl-spiro[cyclohexane-1,1′(1′H)-pyrido[3,4-b]indole]-4-amine citrate (1:1) (trans-diastereoisomer; AMD-6^{trans})

The trans-amide just prepared (188 mg, 0.37 mmol) was dissolved at 80° C. in ethanol (35 ml), and an ethanolic solution (2 ml) of citric acid (77 mg, 0.4 mmol) was added. Stirring was carried out for 2 h at room temperature, crystallisation gradually occurring. The mixture was stored for 1.5 h at 5° C., and the colourless solid was separated off by filtration and washed with diethyl ether (3×3 ml) (146 mg). The filtrate was concentrated and taken up in ethanol (1 ml), and diethyl ether (20 ml) was added. After 16 h, further colourless salt was separated off and washed with diethyl ether (2×2 ml) (36 mg). The two fractions were combined and the trans-citrate AMD-6^{trans} was obtained in a yield of 71% (182 mg) with a melting point of 161-164° C.

¹³C-NMR (101 MHz, DMSO-D₆) δ ppm: (trans diastereoisomer) 22.4, 29.2, 30.7, 37.9, 41.5, 43.1, 58.5, 59.6, 72.0, 105.5, 111.3, 113.2, 113.4, 113.5, 113.8, 117.3, 118.4, 120.5, 122.8, 123.1, 126.5, 127.7, 128.6, 129.1, 129.2, 135.0, 135.6, 139.8, 140.1, 160.7, 163.1, 169.9, 171.2, 175.2

### EXAMPLE AMD-7^{trans}

### 2′,3′,4′,9′-Tetrahydro-N,N-dimethyl-4-(3-fluorophenyl)-2′-(3,4-dimethoxybenzyl)carbonyl-spiro[cyclohexane-1,1′(1′H)-pyrido[3,4-b]indole]-4-amine (trans-diastereoisomer)

3,4-Dimethoxyphenylacetic acid (1 g, 5.1 mmol, 2.2 eq.) is suspended in 25 ml of abs. toluene, and thionyl chloride (0.84 ml, 11.6 mmol, 5.0 eq.) is added. Heating is carried out for 2 h under reflux, and the solvent is then removed. The residue was codistilled with abs. toluene (3×50 ml) and the crude product was dissolved in dichloromethane (37 ml) and transferred to a microwave vessel. Spiroamine AMN-2^{trans} (0.875 mg, 2.32 mmol) and Hünig base (0.78 ml, 580 mmol, 250 eq.) were added, and the microwave vessel was closed and heated for 20 min at 120° C. in a microwave (Initiator Eight, Biotage). For working up, 17 ml of water and 17 ml of 1N sodium hydroxide solution were added to the reaction mixture. This mixture was stirred for 2 h at RT. The phases were then separated and the aqueous phase was extracted 3× with dichloromethane. The combined organic phases were washed with water and dried over sodium sulfate. After the solvent had been removed under reduced pressure, the residue was purified by column chromatography (silica gel; ethyl acetate/n-hexane 2:1). 0.236 g of product AmD-7^{trans} (18%) was obtained.

HPLC/MS analysis: Rₜ=5.45 min; Purity (UV 200-400 nm)>99%; m/z=555.8

### Synthesis of the Cis-Spiroether Comparison Examples (ETHER^{cis})

### EXAMPLE ETHER-1^{cis}

### 6′-Fluoro-4′,9′-dihydro-N,N-dimethyl-4-(3-thienyl)-spiro[cyclohexane-1,1′(3′H)-pyrano[3,4-b]indole]-4-amine, methanesulfonate (2:5) (cis-diastereoisomer)

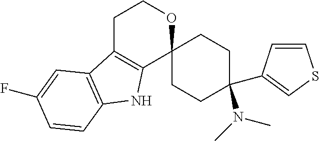 [SMILES: CN(C)[C@]1(c2ccsc2)CC[C@@]2(CC1)OCCc1c3cc(F)ccc3[nH]c12]

The ketone E-5 (446.6 mg, 2 mmol) was dissolved together with 5-fluorotryptophol (2, 394.4 mg, 2 mmol) in absolute 1,2-dichloroethane (30 ml). Methanesulfonic acid (0.13 ml, 2 mmol) was then added to the mixture, whereupon the colour of the reaction solution changed from reddish-brown to dark-grey. After 5 min, a light-grey solid began to precipitate. The batch was stirred for 20 h at RT. Then the methanesulfonate of the cis-spiroether was filtered off with suction and washed with 1,2-dichloroethane (2×10 ml). The light-grey solid was obtained in a yield of 76% (733 mg) and with a melting point of 143-145° C. (ETHER-1^{cis}). 1N NaOH (30 ml) was then added to the filtrate, and stirring was carried out for 2 h at RT. The trans-spiroether thereby precipitated in the form of a colourless solid and was obtained, after filtration, in a yield of 8% (58.5 mg).

¹H NMR (600 MHz, DMSO-d₆): 1.67 (m, 2H) 1.94 (m, 2H) 2.24 (m, 2H) 2.44 (s, 8H) 2.53 (s, 3H) 2.54 (s, 3H) 2.66 (t, J=5.27 Hz, 2H) 2.72 (m, 2H) 3.95 (t, J=5.28 Hz, 2H) 6.84 (m, 1H) 7.14 (m, 1H) 7.19 (dd, J=4.50/8.70 Hz, 1H) 7.47 (d, J=5.10 Hz, 1H) 7.83 (m, 1H) 8.07 (m, 1H) 9.67 (m, 1H) 10.80 (s, 1H)

### EXAMPLE ETHER-2^{cis}

### 4′,9′-Dihydro-N,N-dimethyl-4-(2-thienyl)-spiro[cyclohexane-1,1′(3′H)-pyrano[3,4-b]indole]-4-amine, methanesulfonate (1:2) (cis-diastereoisomer)

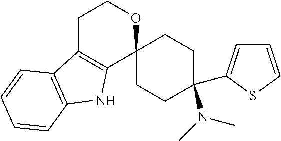 [SMILES: CN(C)[C@]1(c2cccs2)CC[C@@]2(CC1)OCCc1c3ccccc3[nH]c12]

The ketone E-4 (223 mg, 1 mmol) was placed together with tryptophol (2, 161 mg, 1 mmol) in absolute dichloromethane (40 ml). Methanesulfonic acid (0.071 ml, 1.1 mmol) was then added. The mixture was stirred for 16 h at RT, whereupon the methanesulfonate of the spiroether precipitated. The light-grey solid (ETHER-2^{cis}) was filtered off with suction, washed with dichloromethane (2×10 ml) and obtained in a yield of 25% (117 mg) with a melting point of 132° C. 1N NaOH (20 ml) was added to the filtrate, and stirring was carried out for 16 h at RT. The organic phase was separated off and the aqueous phase was extracted with dichloromethane (2×20 ml). The organic phases were combined, dried and concentrated. A substance mixture (274 mg) was obtained and was separated by chromatography [silica gel G (20 g); ethyl acetate/methanol 8:1]. The trans-spiroether was obtained in a yield of 54 (196 mg, m.p. 235-238° C.), and the cis-spiroether was obtained in a yield of 10 (38 mg).

¹H NMR (600 MHz, DMSO-d₆)

1.82 (m, 2H) 1.98 (m, 2H) 2.33 (m, 2H) 2.36 (s, 6H) 2.60 (s, 3H) 2.61 (s, 3H) 2.53 (m, 2H) 2.70 (t, J=5.23 Hz, 2H) 3.96 (t, J=5.23 Hz, 2H) 6.94 (m, 1H) 7.00 (m, 1H) 7.21 (d, J=8.29 Hz, 1H) 7.34 (dd, J=3.74/5.28 Hz, 1H) 7.37 (d, J=7.37 Hz, 1H) 7.59 (d, J=2.76 Hz, 1H) 7.95 (d, J=5.32 Hz, 1H) 9.78 (m, 1H) 10.74 (s, 1H)

Equipment and Methods for HPLC-MS Analysis:

HPLC: Waters Alliance 2795 with PDA Waters 996; MS: ZQ 2000 MassLynx Single Quadrupol MS Detector; Column: Waters Atlantis™ dC18, 3 μm, 2.1×30 mm; Column temperature: 40° C., Eluent A: purified water+0.1% formic acid; Eluent B: acetonitrile (gradient grade)+0.1% formic acid; Gradient: 0% B to 100% B in 8.8 min, 100% B for 0.4 min, 100% B to 0% B in 0.01 min, 0% B for 0.8 min; Flow: 1.0 ml/min; Ionisation: ES+, 25 V; Make up: 100 μl/min 70% methanol+0.2% formic acid; UV: 200-400 nm.

### Study of the Pharmacological Properties of the Example Compounds

A) Comparison of the analgesic effectiveness (as ED₅₀ or % MPE at a specific test dose) in the acute pain model (tail-flick, rat/mouse) and in mononeuropathy pain models (Chung, rat; Bennett, rat) or polyneuropathy pain model (STZ polyneuropathy, rat).

The surprising pharmacological properties of the compounds according to the invention are described primarily by comparing with one another the results from the mononeuropathy pain model according to Chung in the rat and the tail-flick acute pain model in the rat. It is thereby possible to show that the compounds according to the invention do not exhibit a significant anti-nociceptive action in the tail-flick model in the rat at a multiple of the dose which has significant analgesic effectiveness in the Chung model (for example ED₅₀ⁿ). The findings from further models of neuropathic pain, such as the Bennett model in the rat or STZ polyneuropathy in the rat, underline the generally very good effectiveness of the compounds in different forms of neuropathic pain.

### Analgesia Test in the Tail-Flick Test in the Rat

Test Animals:

Female Sprague Dawley rats (crl: CD (SD) outbred; breeder: Charles River, Sulzfeld, Germany); body weight: 130-190 g; the animals are kept in standard cages (type IV Makrolon cages, Ebeco, Castrop-Rauxel, Germany) occupied by in each case not more than 8 animals, with a 12:12 h light/dark rhythm and with food and tap water ad libitum.

Description of the Method:

The analgesic effectiveness of the test compounds was studied in the burning ray (tail-flick) test in the rat according to the method of D'Amour and Smith (J. Pharm. Exp. Ther. 72, 74 79 (1941)). The animals were placed singly into special test cages and the base of the tail was exposed to a focussed heat ray of a lamp (tail-flick type 50/08/1.bc, Labtec, Dr. Hess). The lamp intensity was so adjusted that the time between switching on of the lamp and the sudden pulling away of the tail (withdrawal latency) in untreated animals was 2.5-5 seconds. Before administration of a test compound, the animals were pre-tested twice within 30 minutes and the mean value of those measurements was calculated as the pre-test mean value. Pain measurement was generally carried out 5, 20, 40, 60, 90, 120, 180 and 240 minutes after intravenous administration of test compound or its vehicle. The antinociceptive action was determined as the increase in the withdrawal latency according to the following formula: (% MPE)=[(T₁−T₀)/(T₂−T₀)]×100, where: T₀=control latency period before administration of substance, T₁=latency period after administration of substance, T₂=maximum exposure time to the burning ray (12 seconds), MPE=maximum possible effect.

In test compounds having antinociceptive action, the dose dependency was determined by administering 3-5 logarithmically increasing doses, which included the threshold dose and the maximum effective dose. The half-maximum effective dose (ED₅₀) with corresponding 95% confidence limits was determined by semi-logarithmic regression analysis at the time of maximum action.

Statistical Evaluation:

The group sizes were usually n=10. Variance analysis with repeated measures (repeated measures ANOVA) as well as a post hoc analysis according to Bonferroni were used to test for statistically significant differences in the % MPE data between the dose groups and the vehicle-control groups. The significance level was set at p<0.05.

Tail-Flick with Reduced Burning Ray Intensity in the Rat

Test Animals:

Male Sprague-Dawley rats (breeder: Janvier, Le Genest St. Isle, France); body weight: 200-250 g; the animals are kept in standard cages (type IV Makrolon cages, Ebeco, Castrop-Rauxel, Germany) occupied by in each case not more than 5 animals, with a 12:12 h light/dark rhythm and with food and tap water ad libitum.

Description of the Method:

The modulatory effectiveness of the test substances on acute, noxious thermal stimuli was studied in the burning ray (tail-flick) test in the rat according to the method of D'Amour and Smith (J. Pharm. Exp. Ther. 72, 74 79 (1941)). The animals were accommodated singly in special test compartments and the base of the tail was exposed to a focussed burning ray of an analgesia meter (model 2011, Rhema Labortechnik, Hofheim, Germany). The intensity of the burning ray was so adjusted that the time between switching on of the burning ray and the sudden pulling away of the tail (withdrawal latency) in untreated animals was about 12-13 seconds. Before administration of a substance according to the invention, the withdrawal latency was determined twice at an interval of 5 minutes and the mean value was defined as the control latency period. Measurement of the withdrawal latency of the tail was carried out for the first time 10 minutes after intravenous administration of test compound or its vehicle. When the antinociceptive effect had subsided (after 2-4 hours), measurements were carried out at intervals of 30 minutes up to a maximum of 6.5 hours after administration of substance. The anti- or pro-nociceptive action was determined as the increase or reduction in the withdrawal latency period according to the following formula: (% MPE)=[(T₁−T₀)/(T₂−T₀)]×100, where: T₀=control latency period before administration of substance, T₁=latency period after administration of substance, T₂=maximum exposure time to the burning ray (30 seconds), MPE=maximum possible effect. In test compounds having antinociceptive action, the dose dependency was determined by administering 3-5 logarithmically increasing doses, which included the threshold dose and the maximum effective dose. The half-maximum effective dose (ED₅₀) with corresponding 95% confidence limits was determined by semi-logarithmic regression analysis at the time of maximum action.

Statistical Evaluation:

The group sizes were usually n=10. Variance analysis with repeated measures (repeated measures ANOVA) as well as a post hoc analysis according to Bonferroni were used to test for statistically significant differences in the % MPE data between the dose groups and the vehicle-control groups. The significance level was set at p<0.05.

### Analgesia Test in the Tail-Flick Test in the Mouse

Test Animals:

Male NMRI mice (breeder: Charles River, Sulzfeld, Germany); body weight: 20-25 g; the animals are kept in standard cages (type III Makrolon cages, Ebeco, Castrop-Rauxel, Germany) occupied by in each case not more than 6 animals, with a 12:12 h light/dark rhythm and with food and tap water ad libitum.

Description of the Method:

The analgesic effectiveness of the test compound was studied in the burning ray (tail-flick) test in the mouse according to the method of D'Amour and Smith (J. Pharm. Exp. Ther. 72 74 79 (1941)). The animals were placed singly in special test cages and the base of the tail was exposed to a focussed heat ray of an electric lamp (tail-flick type 55/12/10.fl, Labtec, Dr. Hess). The intensity of the lamp was so adjusted that the time between switching on of the lamp and the sudden pulling away of the tail (withdrawal latency) in untreated animals was 2.5-5 seconds. Before a test compound was administered, the animals were pre-tested twice within 30 minutes and the mean value of those measurements was defined as the pre-test mean value. Pain measurement was generally carried out 20, 40 and 60 minutes after intravenous administration of test compound or its vehicle. The antinociceptive action was determined as the increase in the withdrawal latency period according to the following formula: (% MPE)=[(T₁−T₀)/(T₂−T₀)]×100, where: T₀=control latency period before administration of substance, T₁=latency period after administration of substance, T₂=maximum exposure time to the burning ray (12 seconds), MPE=maximum possible effect. In test compounds having antinociceptive action, the dose dependency was determined by administering 3-5 logarithmically increasing doses, which included the threshold dose and the maximum effective dose. The half-maximum effective dose (ED₅₀) with corresponding 95% confidence limits was determined by semi-logarithmic regression analysis at the time of maximum action.

Statistical Evaluation:

The group sizes were usually n=10. Variance analysis with repeated measures (repeated measures ANOVA) as well as a post hoc analysis according to Bonferroni were used to test for statistically significant differences in the % MPE data between the dose groups and the vehicle-control groups. The significance level was set at p<0.05.

Chung Model: Mononeuropathic Pain after Spinal Nerve Ligation

Test Animals:

Male Sprague Dawley rats (RjHan:SD outbred; breeder: Janvier, Genest St. Isle, France) having a body weight of 140-160 g were kept in standard cages (type IV Makrolon cages, Ebeco, Castrop-Rauxel, Germany) occupied by in each case not more than 8 animals, with a 12:12 h light/dark rhythm and with food and tap water ad libitum. Between delivery of the animals and the operation, an interval of one week was observed. After the operation, the animals were tested several times over a period of 4-5 weeks, a wash-out period of at least one week being observed.

Description of the Model:

Under pentobarbital anaesthesia (Narcoren®, 60 mg/kg i.p., Merial GmbH, Hallbergmoos, Germany), the left L5, L6 spinal nerves were exposed by removing a piece of the paravertebral muscle and part of the left spinous process of the L5 lumbar vertebra. The spinal nerves L5 and L6 were carefully isolated and bound with a tight ligature (NC-silk black, USP 5/0, metric 1, Braun Melsungen AG, Melsungen, Germany) (Kim and Chung 1992). After ligation, muscle and adjacent tissue were sutured and the wound was closed by means of metal staples. After a recovery period of one week, the animals were placed in cages with a wire floor for measurement of the mechanical allodynia. The withdrawal threshold was determined on ipsilateral and/or contralateral rear paws by means of an electronic von Frey filament (Somedic AB, Malmö, Sweden). The median of five stimulations gave a data point. The animals were tested 30 minutes before and at various times after administration of test substance or vehicle solution. The data were determined as maximum possible effect (% MPE) from the pretests of the individual animals (=0% MPE) and the test values of an independent sham control group (=100% MPE). Alternatively, the withdrawal thresholds were indicated in grams. In test compounds having analgesic action, the dose dependency was determined by administering 3-5 logarithmically increasing doses, which included the threshold dose and the maximum effective dose. The half-maximum effective dose (ED₅₀) with corresponding 95% confidence limits was determined by semi-logarithmic regression analysis at the time of maximum action.

Statistical Evaluation:

The group sizes were usually n=10. Variance analysis with repeated measures (repeated measures ANOVA) as well as a post hoc analysis according to Bonferroni were used to test for statistically significant differences in the % MPE data between the dose groups and the vehicle-control groups. The significance level was set at p<0.05.

Reference:

Kim, S. H. and Chung, J. M., An experimental model for peripheral neuropathy produced by segmental spinal nerve ligation in the rat, Pain, 50 (1992) 355-363.

### Bennett Model: Mononeuropathic Pain in the Rat

Test Animals:

Male Sprague Dawley rats (RjHan:SD outbred; breeder: Janvier, Genest St. Isle, France) having a body weight of 140-160 g were kept in standard cages (type IV Makrolon cages, Ebeco, Castrop-Rauxel, Germany) occupied by in each case not more than 8 animals, with a 12:12 h light/dark rhythm and with food and tap water ad libitum. Between delivery of the animals and the operation, an interval of one week was observed. After the operation, the animals were tested several times over a period of 4 weeks, a wash-out period of at least one week being observed.

Description of the Method:

The study of effectiveness in neuropathic pain was carried out in the Bennett model (chronic constriction injury; Bennett and Xie, 1988, Pain 33: 87-107). Under narcorene anaesthesia, the rats were provided with four loose ligatures of the right ischiatic nerve. The animals develop oversensitivity of the paw innervated by the damaged nerve, which is quantified, after a recovery phase of one week, for about four weeks by means of a 4° C. cold metal plate (cold allodynia). The animals are observed on the plate for a period of 2 minutes, and the number of withdrawal reactions of the damaged paw is measured.

Evaluation and Statistics:

Based on the preliminary value before administration of substance, the action of the substance is determined over a period of one hour at four points in time (e.g. 15, 30, 45, 60 minutes after administration) and the resulting area under the curve (AUC) and the inhibition of cold allodynia at the individual measuring points are expressed as percent action relative to the vehicle control (AUC) or the starting value (individual measuring points). The group size is n=10, the significance of an anti-allodynic action (p<0.05) is determined by means of a variance analysis with repeated measures and a post hoc analysis according to Bonferroni.

### STZ Model: Polyneuropathic Pain in the Rat

Test Animals:

Male Sprague Dawley rats (breeder: Janvier, Genest St. Isle, France); body weight 140-160 g; the animals are kept in standard cages (type IV Makrolon cages, Ebeco, Castrop-Rauxel, Germany) occupied by in each case not more than 8 animals, with a 12:12 h light/dark rhythm and with food and tap water ad libitum.

Description of the Method:

In order to induce diabetes, male Sprague Dawley rats were injected intraperitoneally with streptozotocin (STZ, 75 mg/kg). Diabetic rats had a blood glucose level of at least 17 mM one week after STZ injection. Control animals were injected with a vehicle solution. Determination of the mechanical nociceptive stimulus threshold (in grams) was carried out with an algesiometer in the paw pressure test according to Randall & Selitto (1957). In the test, an increasing pressure stimulus was exerted on the dorsal surface of the rear paw and the pressure which ultimately led to the reflex withdrawal of the paw or to vocalisation was recorded. The tests took place three weeks after induction of diabetes. The mechanical nociceptive stimulus threshold was measured before and 15, 30, 45 and 60 minutes after administration of substance to diabetic animals and to control animals.

References:

Randall L O, Selitto J J. A method for measurement of analgesic activity on inflamed tissue. Arch. Int. Pharamcodyn. 1957; 111:409-19

B) Comparison of the analgesically effective dose range in the mononeuropathic pain model (Chung, rat) with the dose range in which opioid-typical side-effects are observed.

The surprising pharmacological properties of the compounds according to the invention are described primarily by comparing with one another the results from the Chung model in the rat (as an example of analgesic effectiveness against neuropathic pain) and the blood gas analysis model in the rat (as an example of respiratory depression as a very serious yet readily quantifiable opioid-typical side-effect). It is thereby possible to show that the compounds according to the invention do not trigger significant respiratory depression in the rat at a multiple of a dose which has significant analgesic activity in the Chung model (for example ED₅₀ⁿ). The findings from further models of opioid-typical side-effects, such as circulatory parameters in the rabbit, gastrointestinal charcoal passage in the mouse, RotaRod test in the mouse, jumping test in the mouse, as well as conditioned place preference in the rat, underline the generally lacking or very slight opioid-typical side-effects of the compounds according to the invention.

Blood Gas Analysis: Method for Arterial pCO₂ and pO₂ Measurement in the Rat

The respiratory depression action of test substances is studied following i.v. administration to awake instrumented rats. The test parameter is the change in the carbon dioxide partial pressure (pCO₂) and the oxygen partial pressure (pO₂) in the arterial blood after administration of substance.

Test Animals:

Male Sprague-Dawley rats (crl: CD (SD) outbred; breeder: Charles River, Sulzfeld, Germany); weight: 250-275 g; the animals are kept singly in standard cages (type II Makrolon cages, Ebeco, Castrop-Rauxel, Germany), with a 12:12 h light/dark rhythm and with food and tap water ad libitum.

Description of the Method:

At least 6 days before administration of the test substance, a PP catheter is implanted into the femoral artery and the jugular vein of the rats, under pentobarbital anaesthesia. The catheters are filled with heparin solution (4000 I.E.) and closed with a wire pin. Administration of the test substance or vehicle is carried out via the venous catheter. Before administration of the substance or vehicle and at defined points in time after administration of the substance or vehicle, the arterial catheter is in each case opened and flushed with about 500 μl of heparin solution. Then about 100 μl of blood are removed from the catheter and taken up by means of a heparinised glass capillary. The catheter is again flushed with heparin solution and closed again. The arterial blood is analysed immediately by means of a blood gas analysis device (ABL 5, Radiometer GmbH, Willich, Germany). After a minimum wash-out period of one week, the animals can be included in the test again.

Test Evaluation and Statistics:

The blood gas analysis device automatically supplies the values for pCO₂ and pO₂ of the blood in mmHg. Effects of the substance on the partial pressure are calculated as percent changes relative to the preliminary values without substance or vehicle. For statistical evaluation, the measured values after administration of the substance and the simultaneous measured values after application of vehicle are compared by means of single-factor variance analysis (one-way ANOVA) and a post hoc analysis according to Dunnett. The significance level was set at p<0.05. The group sizes are usually n=6.

### Cardiovascular Parameters: Method for Measuring Blood Pressure and Cardiac Frequency in the Awake Rabbit

The action of test substances on the cardiovascular system is studied after i.v. administration to awake rabbits with telemetry. The test parameters are the change in the cardiac frequency and arterial blood pressure after administration of substance.

Test Animals:

Female rabbits (New Zealand Whites; breeder: Charles River, Kisslegg, Germany); body weight: about 3-5.5 kg; the animals are kept singly in special rabbit cages (W×D×H=885×775×600 mm; Ebeco, Castrop-Rauxel, Germany), with a 12:12 h light/dark rhythm and with food and tap water ad libitum.

Test Preparation:

At least 21 days before the start of the experiments, a telemetry unit (TL11M2-D70-PCT from DSI, St. Paul, Minn., USA) for measuring blood pressure and electrocardiogram (ECG) is implanted into the animals, under complete anaesthesia (isoflurane 2-3%). The pressure catheter of the telemetry unit is thereby introduced into the A. femoralis and the two bipotential electrodes are fixed subcutaneously in the sternum region or in the region of the upper left thorax wall. The transmitter unit is sewn into a skin pocket in the left flank region of the animals. Recording of the telemetry signals is carried out via receivers of the RMC-1 type (DSI). For data recording, data storage and data processing, the software package Po-Ne-Mah (DSI) is used.

Test Procedure:

Administration of the substance or vehicle is carried out via a venous catheter (V. auricularis). Before administration of the substance or vehicle and at defined points in time after administration of the substance or vehicle, the cardiac frequency and the arterial blood pressure (systolic, diastolic and mean value) are determined directly by means of the calibrated telemetry system and stored electronically. After a minimum wash-out period of one week, the animals can be included in the test again.

Test Evaluation and Statistics:

From the measured values for blood pressure (in mmHg) and cardiac frequency (in beats per min) at the defined points in time, the mean values of 10 successive heart beats are determined in each case. Effects of the substance on the test parameters are calculated as percent changes relative to the preliminary values without substance or vehicle. For statistical evaluation, the measured values after administration of the substance and the simultaneous measured values after administration of vehicle are compared by means of single-factor variance analysis (one-way ANOVA) and a post hoc analysis according to Dunnett. The significance level was set at p<0.05. The group sizes are usually n=6.

### Charcoal Passage Test: Method of Measuring the Gastrointestinal Transit Speed in the Mouse

Test Animals:

Male NMRI mice (breeder: Charles River, Sulzfeld, Germany), body weight: 30-35 g; the animals are kept in standard cages (type IV Makrolon cages, Ebeco, Castrop-Rauxel, Germany) occupied by in each case not more than 18 animals, with a 12:12 h light/dark rhythm and with food and tap water ad libitum.

Description of the Test:

Before the test, the animals are fasted for 20-24 h on wire-grid cage inserts. An active charcoal suspension (10% active charcoal in 0.5% CMC solution; administered volume: 0.1 ml/10 g body weight) is administered orally to the animals as the marker substance for intestinal passage. The test substance or a vehicle solution is then administered intravenously. Two hours after administration of the active charcoal suspension, the animals are sacrificed by gassing with CO₂. The intestinal tract is then removed from the stomach up to and including the caecum and stretched out on a glass plate wetted with 0.9% NaCl solution. The pylorus-caecum distance and the distance travelled by the charcoal suspension (furthest point) are then measured.

Test Evaluation:

In order to determine the relative inhibition of gastrointestinal transit, the quotient distance travelled by the charcoal suspension (in cm)/pylorus-caecum distance (in cm) is formed. It is indicated as % inhibition. For statistical evaluation, the measured values after administration of the substance and the simultaneous measured values after administration of vehicle are compared by means of a single-factor variance analysis (one-way ANOVA) and a post hoc analysis according to Dunnett. The significance level was set at p<0.05. The group sizes are usually n=10.

### Rota-Rod Test: Method for Studying Motor Coordination in the Mouse

Test Animals:

Male CD-1 mice (breeder: Charles River, Sulzfeld, Germany), body weight: 18-25 g; the animals are kept in standard cages (type IV Makrolon cages, Ebeco, Castrop-Rauxel, Germany) occupied by in each case not more than 18 animals, with a 12:12 h light/dark rhythm and with food and tap water ad libitum.

Description of the Method:

For the description of the method see: Kuribara H., Higuchi Y., Tadokoro S. (1977), Effects of central depressants on Rota-Rod and traction performance in mice. Japan. J. Pharmacol. 27, 117-126.

Statistical Evaluation:

For statistical evaluation, the measured values after administration of substance and the simultaneous measured values after administration of vehicle are compared by means of a single-factor variance analysis (one-way ANOVA) and a post hoc analysis according to Dunnett. The significance level was set at p<0.05. The group sizes are usually n=10.

### Jumping Test: Method for Studying the Physical Dependency Potential in the Mouse

Test Animals:

Male NMRI mice (breeder: Charles River, Sulzfeld, Germany), body weight: 20-24 g; the animals are kept in standard cages (type III Makrolon cages, Ebeco, Castrop-Rauxel, Germany) occupied by in each case not more than 6 animals, with a 12:12 h light/dark rhythm and with food and tap water ad libitum.

Description of the Method:

The test substances are administered intraperitoneally a total of 7× over two days. 5 administrations are carried out on the first day at 9:00, 10:00, 11:00, 13:00 and 15:00 and on the second day at 9:00 and 11:00. The first 3 administrations are given in increasing doses (dosage scheme) and then further at the dose of the third. Withdrawal is precipitated with naloxone 30 mg/kg (i.p.) 2 hours after the last administration of substance. Immediately thereafter, the animals are placed singly in transparent observation boxes (height 40 cm, diameter 15 cm) and the jumping reactions are counted over a period of 15 minutes at 5-minute intervals. Morphine is administered concomitantly in a dose as comparison/standard. Quantification of the withdrawal is made via the number of jumps 0 to 10 min after naloxone administration. The number of animals per group with more than 10 jumps/10 minutes is determined and documented as “% positive animals”. In addition, the average jumping frequency in the group is calculated.

Statistical Evaluation:

The evaluation of the experimental findings in respect of statistically significant differences between the dose groups and the vehicle-control groups is preferably carried out by means of Fisher's exact test for the parameter “% positive animals” and by means of the Kruskal-Wallis test for the parameter “jumping frequency”, preferably as described in the experimental section. The significance level is set at p<0.05 in each case. The group sizes are usually n=12.

Reference:

Saelens J K, Arch Int Pharmacodyn 190: 213-218, 1971

### Conditioned Place Preference: Method for Studying the Possible Induction of Mental Dependency/Addiction in the Rat

Description of the Method:

For the study of place preference see: Tzschentke, T. M., Bruckmann, W. and Friderichs, F. (2002) Lack of sensitization during place conditioning in rats is consistent with the low abuse potential of tramadol. Neuroscience Letters 329, 25-28.

Statistical Evaluation:

The evaluation of the experimental findings in respect of statistically significant differences in the animals' preference for the active ingredient or the vehicle is preferably carried out by means of a paired t-test. The significance level is set at p<0.05. The group sizes are usually n=8.

TABLE 1a
Summary of the pharmacological data for AMD-6^{cis}

| | | | Difference |
|---|---|---|---|
| Test system | Measured parameter | Findings ¹ | factor ² |
| ORL1 receptor | Binding affinity | Ki = 0.030 μM | — |
| binding | | | |
| μ-Opioid | Binding affinity | Ki = 0.138 μM | — |
| receptor binding | | | |
| Chung, rat | Inhibition of neuropathic pain | ED₅₀ = 9 μg/kg i.v.; up to the | — |
| | in mononeuropathy | highest test dose (21.5 μg/kg | |
| | (separation of anti-allodynic | i.v.): No antinociceptive | |
| | and antinociceptive action) | action in healthy tissue. | |
| Bennett, rat | Inhibition of neuropathic pain | ED₅₀ = 7 μg/kg i.v. | — |
| | in mononeuropathy | | |
| STZ, rat | Inhibition of neuropathic pain | ED₅₀ about 1 μg/kg i.v.; up to | — |
| | in diabetic polyneuropathy | the highest test dose | |
| | | (10 μg/kg i.v.): No | |
| | | antinociceptive action in | |
| | | neuropathic control animals. | |
| Tail-flick, rat | Inhibition of acute pain | NOEL: 1 mg/kg i.v. or 4.64 | 220-1000x |
| | (nociceptive pain) | mg/kg i.v. at reduced burning | |
| | | ray intensity | |
| Blood gas | Respiratory depression | NOEL: 1 mg/kg i.v. | 220x |
| analysis, rat | measured as increase in | | |
| | arterial pCO₂ and fall in | | |
| | arterial pO₂ | | |
| Cardiovascular | Arterial blood pressure and | NOEL: 1 mg/kg i.v. | 220x |
| system, rabbit | cardiac frequency | | |
| Charcoal | Gastrointestinal transit | NOEL: 3 mg/kg i.v. | 660x |
| passage, mouse | | | |
| RotaRod test, | Motor coordination | NOEL: ≧10 mg/kg i.v. | >2200x |
| mouse | | | |
| Jumping test, | Physical dependency/ | NOEL: 10 mg/kg i.p. | 2200x |
| mouse | withdrawal symptoms | | |
| Place | Mental dependency | NOEL: ≧13.8 mg/kg i.p. | >3000x |
| preference, rat | | | |

¹ NOEL (=No Observed Effect Level) denotes the upper dose without findings (i.e. dose without significant effect)
² The difference factors were calculated as the quotient of NOEL and a mean ED₅₀ⁿ from the neuropathy models (here: 4.5 μg/kg)

TABLE 1b
Summary of the pharmacological data for AMD-7^{cis}

| | | | Difference |
|---|---|---|---|
| Test system | Measured parameter | Findings ¹ | factor ² |
| ORL1 receptor | Binding affinity | Ki = 0.070 μM | — |
| binding | | | |
| μ-Opioid | Binding affinity | Ki = 0.450 μM | — |
| receptor binding | | | |
| Chung, rat | Inhibition of neuropathic pain | ED₅₀ = 88 μg/kg i.v.; no | — |
| | in mononeuropathy | antinociceptive action in | |
| | (separation of anti-allodynic | healthy tissue. (Test dose: | |
| | and antinociceptive action) | 100 μg/kg i.v.) | |
| STZ, mouse | Inhibition of neuropathic pain | 68% MPE at 100 μg/kg i.p.; | — |
| | in diabetic polyneuropathy | no antinociceptive action in | |
| | | non-neuropathic control | |
| | | animals. | |
| Tail-flick, rat | Inhibition of acute pain | NOEL: ≧10 mg/kg i.v. | >110x |
| | (nociceptive pain) | | |
| Blood gas | Respiratory depression | NOEL: 1 mg/kg i.v. | 11x |
| analysis, rat | measured as increase in | | |
| | arterial pCO₂ and fall in | | |
| | arterial pO₂ | | |
| Circulatory | Arterial blood pressure and | NOEL: ≧3 mg/kg i.v. | >34x |
| system, rabbit | cardiac frequency | | |
| Charcoal | Gastrointestinal transit | NOEL: 1 mg/kg i.v. | 11x |
| passage, mouse | | | |
| RotaRod test, | Motor coordination | NOEL: 10 mg/kg i.v. | 110x |
| mouse | | | |
| Jumping test, | Physical dependency/ | NOEL: ≧10 mg/kg i.p. | >110x |
| mouse | withdrawal symptoms | | |
| Place | Mental dependency | NOEL: ≧20 mg/kg i.p. | >220x |
| preference, rat | | | |

¹ MPE (=Maximum Possible Effect) denotes the maximum possible effect; NOEL (=No Observed Effect Level) denotes the upper dose without findings (i.e. dose without significant effect)
² The difference factors were calculated as the quotient of NOEL and a mean ED₅₀ⁿ from the neuropathy models (here: 88 μg/kg)

Conclusion:

Examples AMD-6^{cis} and AMD-7^{cis} were chosen to illustrate the surprising pharmacological properties of the compounds according to the invention. These are high-affinity ORL1 receptor and μ-opioid receptor ligands having a ratio of ORL1 receptor affinity to μ-opioid receptor affinity of about 5 or about 6. Examples AMD-6^{cis} and AMD-7^{cis} show that the compounds according to the invention have very high effectiveness against neuropathic pain (here: ED₅₀ⁿ between 1 and 10 μg/kg i.v. or 88 μg/kg i.v.). In the acute pain model, on the other hand, even at doses which were from 100 to 1000 times higher than the effective doses in the neuropathy model, no significant antinociceptive action was observed. Likewise, in animal models for studying side-effects, no significant opioid-type side-effects (such as respiratory depression, reduction of blood pressure and cardiac frequency, constipation, central nervous effects, physical dependency, mental dependency/addiction) were observed at doses which were from 11 to more than 3000 times higher.

TABLE 2
Overview of selected pharmacological or pharmacokinetic
characteristics of further examples

| | | | | | | | | t_{1/2}, rat, |
|---|---|---|---|---|---|---|---|---|
| | | | | | | | | 100 μg/kg, |
| | | | | | | | | i.v.// |
| | | | | | | | | pharmaco- |
| | | | | | Blood | | | dynamic |
| | Ki | Ki | | Tail- | gas | Charcoal | | duration |
| | (ORL₁) | (μ) | Chung, | flick, | analysis, | passage, | RotaRod, | of action |
| Compound | [μM] | [μM] | rat | rat | rat | mouse | mouse | (dose) |
| AMD-6^{cis} | 0.030 | 0.138 | ED₅₀ = | NOEL² = | NOEL = | NOEL = | NOEL: | 8 h//>>5 h |
| | | | 9 μg/kg | 1000 | 1000 | 3000 | ≧10000 | (10 μg/kg |
| | | | i.v. | μg/kg | μg/kg | μg/kg | μg/kg | i.v.) |
| | | | | i.v. | i.v. | i.v. | i.v. | |
| AMD-1^{cis} | 0.018 | 0.032 | 18% MPE | NOEL > | NOEL = | Not | Not | not determined// |
| | | | at 100 | 100 | 1000 | carried | carried | not determined |
| | | | μg/kg | μg/kg | μg/kg | out | out | |
| | | | i.v. | i.v. | i.v. | | | |
| AMD-2^{cis} | 0.017 | 0.05 | 35% MPE | NOEL > | Not | NOEL = | Not | not determined// |
| | | | at 100 | 1000 | carried | 4600 | carried | about 3 h |
| | | | μg/kg | μg/kg | out | μg/kg | out | (100 μg/kg |
| | | | i.v. | i.v. | | i.v. | | i.v.) |
| AMD-3^{cis} | 0.016 | 0.059 | 42% MPE | NOEL > | Not | NOEL = | NOEL: | not determined// |
| | | | at 100 | 1000 | carried | 3000 | ≧10000 | about 3 h |
| | | | μg/kg | μg/kg | out | μg/kg | μg/kg | (100 μg/kg |
| | | | i.v. | i.v. | | i.v. | i.v. | i.v.) |
| AMD-4^{cis} | 0.003 | 0.009 | 20% MPE | NOEL > | NOEL = | Not | Not | not determined// |
| | | | at 100 | 100 | 300 | carried | carried | 1-3 h |
| | | | μg/kg | μg/kg | μg/kg | out | out | (100 μg/kg |
| | | | i.v. | i.v. | i.v. | | | i.v.) |
| AMD-7^{cis} | 0.070 | 0.450 | ED₅₀ = | NOEL ≧ | NOEL = | NOEL = | NOEL = | 3 h//about 3 h |
| | | | 88 | 10000 | 1000 | 1000 | 10000 | (100 μg/kg |
| | | | μg/kg | μg/kg | μg/kg | μg/kg | μg/kg | i.v.) |
| | | | i.v. | i.v. | i.v. | i.v. | i.v. | |

¹ORL1/μ affinity ratio defined as 1/[K_{i(ORL1)}/K_{i(μ)}]
²NOEL (= No Observed Effect Level) denotes the upper dose without findings (i.e. dose without significant effect)

Conclusion:

The compounds according to the invention exhibit very good effectiveness against neuropathic pain. Surprisingly, on the other hand, no significant antinociceptive action was observed in the acute pain model even at doses which were about 10 times to more than 100 times higher than the effective doses in the neuropathy model. Likewise, no significant opioid-typical side-effects were observed, surprisingly, in the side-effect animal models (e.g. blood gas analysis, gastrointestinal charcoal passage and RotaRod test) at 10 times to more than 300 times higher doses.

TABLE 3
Comparison of cis- and trans-spiroamine

| | Ki | | | | |
|---|---|---|---|---|---|
| | (ORL₁) | Ki (μ) | | | Blood gas analysis, |
| Compound | [μM] | [μM] | Chung, rat | Tail-flick, rat | rat |
| AMD-6^{cis} | 0.030 | 0.138 | ED₅₀ = 9 μg/kg i.v. | NOEL ² = 1000 μg/kg i.v. | NOEL = 1000 μg/kg i.v. |
| AMD-6^{trans} | 0.002 | 0.008 | NOEL ≧ 100 μg/kg | ED₅₀ = 640 μg/kg i.v. | NOEL = 300 μg/kg i.v. |
| | | | i.v | | |
| AMD-7^{cis} | 0.070 | 0.450 | ED₅₀ = 88 μg/kg i.v. | NOEL ² ≧ 10000 μg/kg i.v. | NOEL = 1000 μg/kg i.v. |
| AMD-7^{trans} | 0.001 | 0.001 | Not carried out | 54% MPE at | Not carried out |
| | | | | 31.6 μg/kg i.v. | |
| AMN-2^{cis} | 0.012 | 0.031 | ED₅₀ = 895 μg/kg i.v. | NOEL = 1000 μg/kg i.v. | Not carried out |
| AMN-2^{trans} | 0.0004 | 0.0005 | 27% MPE at | 60% MPE at | Not carried out |
| | | | 30 μg/kg i.v. | 100 μg/kg i.v. | |

¹ ORL1/μ affinity ratio defined as 1/[K_{i(ORL1)}/K_{i(μ)}]
² NOEL (=No Observed Effect Level) denotes the upper dose without findings (i.e. dose without significant effect)

Conclusion:

Surprisingly, only the cis-spiroamines according to the invention (here Examples AMD-6^{cis} and Example AMN-2^{cis}) exhibit good effectiveness against neuropathic pain while at the same having no antinociceptive action in acute pain. Likewise, no significant opioid-typical side-effects are observed in the side-effect animal models (as an example here blood gas analysis) at doses that are higher by a multiple. The trans-spiroamines (here Comparison Example AMD-6^{trans} and Comparison Example AMN-2^{trans}), on the other hand, show no difference between doses that are effective against neuropathic pain or against acute pain. Likewise, no difference between doses at which opioid-typical side-effects (as an example here blood gas analysis) occur is observed. In the overall comparison, AMD-5^{cis} and AMD-6^{cis} show the greatest differences with the highest possible analgesic action.

TABLE 4
Comparison of cis-spiroamines and cis-spiroethers

| | Ki | | | |
|---|---|---|---|---|
| | (ORL₁) | Ki (μ) | | Tail-flick, rat or |
| Compound | [μM] | [μM] | Chung, rat | mouse* |
| AMN-2^{cis} | 0.012 | 0.031 | ED₅₀ = | NOEL ² = |
| | | | 895 μg/kg i.v. | 1000 μg/kg i.v. |
| Ether-2^{cis} | 0.031 | 0.092 | 17% MPE at | 78% MPE at |
| | | | 100 μg/kg i.v | 1000 μg/kg i.v.* |
| Ether-1^{cis} | 0.06 | 0.12 | 28% MPE at | 33% MPE at |
| | | | 100 μg/kg i.v | 1000 μg/kg i.v. |

¹ ORL1/μ affinity ratio defined as 1/[K_{i(ORL1)}/K_{i(μ)}]
² NOEL (=No Observed Effect Level) denotes the upper dose without findings (i.e. dose without significant effect)

Conclusion:

Surprisingly, only the cis-spiroamines according to the invention (here Example AMN-2^{cis}) exhibit good effectiveness against neuropathic pain while at the same having no antinociceptive action in acute pain. Likewise, no significant opioid-typical side-effects are observed in the side-effect animal models (as an example here blood gas analysis) at doses that are higher by a multiple. Cis-spiroethers (here Comparison Example Ether-2^{6s} and Comparison Example Ether-1^{6s}), on the other hand, show no marked differences between doses that are effective against neuropathic pain or against acute pain.

TABLE 5
Comparison of AMD-5^{cis} (free base) and AMD-6^{cis} (citrate salt)

| | Ki | | | |
|---|---|---|---|---|
| | (ORL₁) | Ki (μ) | | |
| Compound | [μM] | [μM] | Chung, rat | Tail-flick, rat |
| AMD-5^{cis} | 0.030 | 0.138 | ED₅₀ = | NOEL ² = |
| | | | 17 μg/kg i.v. | 30000 μg/kg i.p. |
| AMD-6^{cis} | 0.020 | 0.117 | ED₅₀ = | NOEL > |
| | | | 9 μg/kg i.v. | 10000 μg/kg i.v. |

¹ ORL1/μ affinity ratio defined as 1/[K_{i(ORL1)}/K_{i(μ)}]
² NOEL (=No Observed Effect Level) denotes the highest dose without findings (i.e. dose without significant effect)

Conclusion:

A comparison of AMD-5^{cis} (free base) and AMD-6^{cis} (citrate salt) revealed no relevant differences in the pharmacological properties of the base and the salt.

TABLE 6
Comparison of the affinities towards individual receptors

| | | | | | | | | | Pain, rat | |
|---|---|---|---|---|---|---|---|---|---|---|
| | | | | | | | | | | neuro- |
| | | | | | | | | | Acute | pathic |
| | | | | | | | | | (tail | (SNL |
| | | | | | | | | | flick) | [Chung]) |
| | ORL1 | | μ-Opioid | | k-Opioid | | d-Opioid | | ED_{50 rat} [μg/kg] | |
| | Ki * | EC₅₀ ** | Ki | EC₅₀ | Ki | EC₅₀ | Ki | EC₅₀ | % MPE (@ μg/kg) | |
| AMD-3^{cis} | 16 | 102/ | 59 | 1112/ | 160 | 874/ | 6.7 | 41/ | 0% | 106 |
| | | 92% | | 82% | | 42% | | 92% | (1000) | |
| | | | | | | | | | 73% | |
| | | | | | | | | | (10000) | |
| AMD-3^{trans} | 14 | 16/ | 12 | 13/ | 49 | —/ | 8 | —/ | 0% | 20% |
| | | 81% | | 66% | | 55% | | 87% | (1000) | (100) |
| AMD-5^{cis} | 30 | 76/ | 138 | 300/ | 768 | 1035/ | 38 | 463/ | 0% | 9.2 |
| | | 106% | | 63% | | 30% | | 78% | (1000) | |
| | | | | | | | | | 58% | |
| | | | | | | | | | (10000) | |
| AMD-6^{trans} | 3 | 47/ | 8 | 79/ | 19 | 59/ | 6 | 19/ | 640 | 400 |
| | | 104% | | 97% | | 88% | | 126% | | |
| AMD-7^{cis} | 70 | 50/ | 450 | 49/ | 542 | 1170/ | 791 | 2684/ | 0% | 88 |
| | | 90% | | 94% | | 85% | | 106% | (10000) | |
| AMD-7^{trans} | 1 | 16/ | 1 | 3/ | 4 | 29/ | 1 | 5/ | 54% | not |
| | | 90% | | 88% | | 64% | | 82% | (31.6) | carried |
| | | | | | | | | | | out |

* Radio-binding assay-Ki in nM
** GTPgammaS assay-EC50 in nM and relative efficacy in %
Ki [nM]
EC₅₀ [eff. %]

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1.-20. (canceled)

21. A compound of formula I: 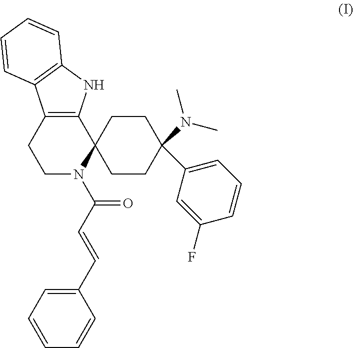 [SMILES: CN(C)[C@]1(c2cccc(F)c2)CC[C@]2(CC1)c1[nH]c3ccccc3c1CCN2C(=O)/C=C/c1ccccc1]
or a physiologically acceptable salt thereof,
wherein the compound exhibits at least 90% d.e.

22. A pharmaceutical composition comprising a compound as in claim 21 and a physiologically acceptable carrier.

23. A pharmaceutical composition as in claim 22, wherein the composition is solid, liquid, or pasty.

24. A pharmaceutical composition as in claim 22, wherein the composition comprises the compound in an amount of 0.001 to 99 wt. %, based on the total weight of the composition.

25. A pharmaceutical dosage form comprising a pharmaceutical composition as in claim 22, wherein the dosage form is a controlled release dosage form adapted for administration not more than once daily.

26. A pharmaceutical dosage form comprising a pharmaceutical composition as in claim 22, wherein the dosage form is suitable for systemic administration.

27. A pharmaceutical dosage form as in claim 26, wherein the dosage form is suitable for oral administration.

28. A pharmaceutical dosage form as in claim 25, wherein the dosage form contains the compound in an amount insufficient to effectively treat acute pain.

29. A pharmaceutical dosage form as in claim 28, wherein the dosage form comprises the compound in a dose in a range of 1.0 μg to 10 μg, based on the molecular weight of the free base.

30. A method of treating chronic pain in a subject in need thereof, the method comprising administering to the subject an effective chronic pain alleviating amount of a compound as in claim 21.

31. A method as in claim 30, wherein the chronic pain is selected from the group consisting of inflammatory pain, visceral pain, tumour pain and neuropathic pain.

32. A method as in claim 30, wherein the chronic pain is neuropathic pain of mononeuropathic/neuralgic or polyneuropathic origin.

33. A method as in claim 30, wherein the compound is administered to the subject not more than once daily.

34. A method as in claim 30, wherein the compound is administered in an amount insufficient to effectively treat acute pain.
